(19) United States
(12) Patent Application Publication
(10) Pub. No.: US 2010/0286778 A1
(43) Pub. Date: Nov. 11, 2010

(54) Textile-Based Spinal Implant and Related Methods

(76) Inventors: Lukas Eisermann, San Diego, CA (US); Alan McLeod, Somerset (GB); Christopher Reah, Taunton (GB); Peter Butcher, Nottingham (GB); Eugene Shoshtaev, San Diego, CA (US); Keith Valentine, San Diego, CA (US)

Correspondence Address: NuVasive;c/o CPA Global, P.O. Box 52050, Minneapolis, MN, 55402, US

(21) Appl. No.: 12/596,657

(22) PCT Filed: Apr. 18, 2008
(86) PCT No.: PCT/US08/60944

§ 371 Date: Jul. 26, 2010

Related U.S. Application Data
(60) Provisional application No. 60/925,205, filed on Apr. 18, 2007.
(60) Provisional application No. 60/966,988, filed on Aug. 31, 2007.
(60) Provisional application No. 61/008,736, filed on Dec. 21, 2007.

(51) Int. Cl. A61F 2/44

(52) U.S. Cl. 623/17.11

(57) ABSTRACT

A textile-based surgical implant that can be inserted through any number of suitable surgical approaches, including but not limited to lateral, anterior, anterior-lateral, posterolateral, and/or posterior approaches. When applied to spine surgery and inserted into an intervertebral disc space, the implant restores the normal height of the intervertebral disc space, while advantageously preserving the natural motion of the spine. The textile construction of the implant is beneficial because it is generally compliant and thereby restores and/or improves spinal motion. The compliant nature of the textile-based implant provides the required flexibility and elasticity to advantageously support physiological movements, as opposed to fusion surgery which forms a boney bridge between adjacent vertebral bodies. In addition, the porosity and biocompatibility of the textile-based implant facilitates tissue ingrowth throughout part or all of the implant, which helps to secure and encapsulate the implant in the intervertebral space.

### CROSS REFERENCES TO RELATED APPLICATIONS

The present application is an international patent application claiming benefit under 35 U.S.C. §119(e) from U.S. Provisional Application Ser. No. 60/925,205, filed on Apr. 18, 2007, U.S. Provisional Application Ser. No. 60/966,988, filed on Aug. 31, 2007, and U.S. Provisional Application Ser. No. 61/008,736, filed Dec. 21, 2007, the entire contents of which are hereby expressly incorporated by reference into this disclosure as if set forth fully herein. The present application also incorporates by reference the following commonly-owned and co-pending applications in their entireties: PCT Application Serial No. PCT/US2006/021814, entitled “Improvements In and Relating to Implants,” filed on Jun. 5, 2006; PCT/US2008/052524, entitled “Using Zigzags to Create Three-Dimensional Embroidered Structures,” filed on Jan. 30, 2008; and PCT Application Serial No. PCT/US2008/053315, entitled “Medical Implants with Pre-Settled Cores and Related Methods,” filed on Feb. 7, 2008.

### BACKGROUND OF THE INVENTION

I. Field of the Invention

The present invention relates generally to surgical implants and, more particularly, to textile-based implants for surgical implantation and related methods of manufacture and use.

II. Discussion of the Prior Art

Surgical implants exist for a myriad of clinical needs, including spine surgery to treat diseased or damaged intervertebral discs. Increasingly, this treatment involves replacing all or part of the disc with a prosthetic disc rather than fusing the adjacent vertebrae. A wide variety of designs of disc prostheses exist. Disc prostheses based on either articulating metal plates or metal end plates supporting a polyethylene spacer are now in clinical use. These mechanical total disc replacements help to reduce the loss in spinal mobility and the degeneration of adjacent discs commonly associated with fusion.

While mechanical total disc replacements are a great improvement over fusion, some surgeons would rather use non-mechanical motion preserving implants. Previously, there has been developed a textile-based total disc replacement having a textile-based core provided in a textile retaining jacket. An example of one such implant is described in the above-referenced PCT Application No. PCT/US2006/021814. The textile-based implant is advantageous in that it allows tissue ingrowth and is generally compliant and therefore is capable of restoring disc height and preserving the motion of the spinal unit. While such textile-based motion preserving spinal implants show great promise, there is still room for improvement.

The present invention is directed to improving textile-based implants, including but not limited to textile-based motion preserving spinal implants.

### SUMMARY OF THE INVENTION

The present invention accomplishes this goal by providing a textile-based surgical implant that can be inserted through any number of suitable surgical approaches, including but not limited to lateral, anterior, antero-lateral, postero-lateral, and/or posterior approaches. An implant according to the present invention is suitable for use in a variety of surgical applications, including but not limited to spine surgery. When applied to spinal surgery and inserted into an intervertebral disc space, the implant restores the normal height of the intervertebral disc space, while advantageously preserving the natural motion of the spine. The textile construction of the implant is beneficial because it is generally compliant and thereby restores and/or improves spinal motion. The compliant nature of the textile-based implant provides the required flexibility and elasticity to advantageously support physiological movements, as opposed to fusion surgery which forms a boney bridge between adjacent vertebral bodies. In addition, the porosity and biocompatibility of the textile-based implant facilitates tissue ingrowth throughout part or all of the implant, which helps to secure and encapsulate the implant in the intervertebral space.

According to one exemplary embodiment of the present invention, the implant may include a spacer disposed within an encapsulating jacket. The jacket may be constructed from any of a variety of textile materials (e.g. polyester fiber, polyethylene (including ultra high molecular weight polyethylene), polyclycolic acid, polylactic acid, etc.) via any number of textile processing techniques (e.g. embroidery, weaving, three-dimensional weaving, knitting, three-dimensional knitting, injection molding, compression molding, cutting woven or knitted fabrics, etc.). The jacket may encapsulate the spacer fully (i.e. disposed about all surfaces of the spacer) or partially (i.e. with one or more apertures formed in the jacket allowing direct access to the spacer). The various layers and/or components of the spacer may be attached or unattached to the encapsulating jacket. The jacket may optionally include one or more fixation elements for retaining the jacket in position after implantation, including but to limited to one or more flanges extending from or otherwise coupled to the jacket and screws or other affixation elements (e.g. nails, staples sutures, tacks, adhesives, etc.) to secure the flange to an adjacent anatomical structure (e.g. vertebral body). This may be facilitated by providing one or more apertures within the flange dimensioned to receive the screws or other fixation elements.

Although generally described herein as a two-part construct (with a spacer within an encapsulating jacket) it will be appreciated that the textile-based surgical implant may include a spacer without the encapsulating jacket, wherein the shape of the spacer is maintained through any number of suitable techniques, including but not limited to stitching the spacer together via supplemental stitches. It will also be appreciated that the implant may be comprised of a spacer and an encapsulating jacket made from a single continuous embroidery. In all instances, the implant provides the necessary height and shape to restore the natural curvature of the spine, along with the required flexibility and elasticity to partially or fully restore or improve the physiologic movements of the spine.

A variety of embodiments may be used in constructing the implant of the present invention. According to one example of a method for creating an implant according to the present invention, the first step involves manufacturing a base textile structure used to form the spacer. The base textile structure is preferably manufactured via an embroidery process using any number of biocompatible filament materials (including but not limited to polyester thread). This embroidery process may or may not include a backing layer. For example, stitches may be added onto a flat fabric, i.e. backing layer or base cloth. Subsequently, the backing layer may be dissolved. It will be appreciated that the base textile structure may be formed from different materials and/or made from techniques other than embroidery without departing from the scope of the present invention.

By way of example only, the base textile structure may be comprised of a plurality of hinged embroidered layer regions. The layer regions of the base textile structure may be connected together and separated by a distance to form a plurality of hinge regions between the layer regions. The base textile structure is then folded to form the spacer and placed within the encapsulating jacket. It will be appreciated that the folding process may be performed in any number of manners without departing from the scope of the present invention. In all instances, the implant restores the height of the intervertebral disc space, while advantageously preserving the natural motion of the spine. The layer regions may be coupled together without the use of hinge regions, for example, via the use of other fixation or coupling mechanisms, including but not limited to supplemental stitching and/or adhesives capable of coupling adjacent or multiple layer regions together.

After the layer regions of the spacer are folded but before implantation, the stack of layer regions that comprise the spacer may be pre-settled, similar to the pre-settling process shown and described in commonly owned and co-pending PCT Application No. PCT/US2008/053315, referenced above. During the pre-settling process, the stack of layer regions is compressed under a high load in order to make the implant more dimensionally stable and compact. Once implanted and used to restore the intervertebral disc height, the pre-settled stack of layer regions that comprise the spacer will be more likely to keep its shape while sustaining the axial load and pressure from the adjacent vertebrae. The spacer may be pre-settled before or after placement into the encapsulating jacket.

As opposed to a rigid block used for fusion, the stack of layer regions advantageously allows for natural motion, providing the ability to accommodate bending movements while simultaneously preserving the overall disc height. The stack of layer regions compresses and fans out according to the pressures to which it is subjected. When the implant is being compressed between the adjacent vertebrae, the stack of layer regions of the spacer is very stable and offers resistance to the compounding load, thus maintaining the disc height. When the implant undergoes flexion, the stack of layer regions can react with some flexibility yet still maintain the disc height.

During bending, the individual layer regions can expand by fanning-out or opening out slightly. The hinge regions help facilitate bending movements by holding the layer regions together and, at the same time, allowing for the accordion-like opening between the layer regions as the implant is flexed. Consequently, the implant conforms to the shape of the intervertebral space into which it is inserted because of the potential for the layer regions to be able to separate. By allowing mobility while preventing overall height loss, the stack of layer regions that comprise the spacer helps approximate the natural motion of the spine.

It will be appreciated that the number of layer regions may be increased or decreased without departing from the scope of the present invention. This may be done for any number of different purposes, including but not limited to varying the height of the spacer. The spacer may also be provided in any number of suitable dimensions depending upon the surgical application and patient pathology. The spacer may take many shapes and any of a variety of shapes are contemplated for the spacer. For example, the spacer may be any of generally square, rectangular, spherical, wedge, oval, elliptical, trapezoidal, and polygonal shape. The spacer may have a generally flat upper surface and a generally flat lower surface, or one or both surfaces may be convex or concave. One of the upper and lower surfaces of the spacer may be convex and the other surface may be concave. In addition, vertically oriented holes or apertures running from the top of the spacer to the bottom of the spacer may be included in order to facilitate the spread and nourishment of tissue ingrowth.

A variety of features may be incorporated into the spacer to match (or approximate) the natural curvature of the spine and thereby support the full range of physiological movements. For example, each single layer region of the spacer may be contoured such that when the individual layer regions are folded and stacked on top of one another the spacer achieves an anatomical dome shape for the top and bottom of the implant. By doing so, the implant would mimic the anatomical shape of an intervertebral disc. In addition, each single layer region of the spacer may be contoured to achieve an overall tapered shape. Thus, when the individual layer regions are folded and stacked on top of one another, the overall shape of the spacer matches the natural lordotic and/or kyphotic angles in any given region of the spine (i.e. lordosis in the cervical and lumbar regions of the spine and kyphosis in the thoracic region of the spine.)

This means that the spacer may have a cross-sectional shape in which the vertebral-contacting surfaces are not parallel. This includes (but is not limited to) cross-sectional shapes wherein the vertebral-contacting surfaces are partially angled relative to one another (e.g. along a portion of the cross-section), fully angled relative to one another (e.g. along all or a substantial portion of the cross-section), generally angled relative to one another (e.g. one or more generally flat regions arranged in a decreasing or increasing “stair step” fashion), curved (e.g. partially, fully, generally, and/or slightly) relative to one another, and/or any combination of the above.

The individual layer regions of the spacer may be contoured in different ways to replicate the anatomical shape of an intervertebral disc. According to a first exemplary method for adding contour to a single layer region of the spacer, a layer region may be repeatedly overstitched in different sections using thread of the same thickness. According to a second exemplary method for adding contour to a single layer region of the spacer, threads of varying thickness or diameter are used to form sections having different height. According to a third exemplary method for adding contour to a single layer region of the spacer, fabric layers of different shapes are stacked consecutively on top of one another to form sections having different height. The key is that layer region results in a textile layer having a contoured cross-sectional shape (e.g. tapered or dome-shaped) due to the sections of varying height that are formed. Thus, if the spacer contains contoured layer regions, then the spacer will have a contoured cross-sectional shape as a result of the individual contoured layer regions building the height of the spacer when folded and stacked on top of one another. In this way, the overall shape of the spacer replicates the anatomical shape of an intervertebral disc, thereby matching the natural curvature of the spine.

In some instances, it may be advantageous that each single layer region of the spacer be contoured to achieve an overall tapered shape. Although described herein largely in terms of restoring lordosis in the lumbar or cervical spinal regions (i.e. with the implant height greater at the anterior region of the disc space than in the posterior region of the disc space), it will be understood that the taper may also be reversed to match the kyphotic curvature of the thoracic spine. It will also be understood that the term “taper” is defined to mean any cross-sectional shape in which the vertebral-contacting surfaces of the textile-based implant of the present invention are not parallel, as defined above.

According to a first exemplary method for achieving a tapered textile-based core, threads of different thicknesses are used to form regions of fabric having different height. According to a second exemplary method for achieving a tapered textile-based core, more layers of fabric are used where more height is needed and fewer layers of fabric are used where less height is needed. According to a third exemplary method for achieving a tapered textile-based core, sections of different densities are used to form regions of fabric having different height. In addition, modifying the third exemplary method to create varying stitching densities may produce a smoother gradient profile in the taper angle. According to a fourth exemplary method for achieving a tapered textile-based core, sections of different densities are created through a difference in yarn spacing. According to a fifth exemplary method for achieving a tapered textile-based core, the width and length of various layers of fabric increases gradually such that they form a rectangular pyramid shape when stacked upon one another. The stack of layers is then situated vertically to form the tapered effect of the textile-based core.

In one embodiment of the present invention, the spacer contains one or more internal radio-opaque markers. The radio-opaque marker may be composed of any kind of radio-opaque material including but not limited to metal (e.g. titanium, stainless steel, etc.). Radio-opaque markers are advantageous when tracking the implant post-surgery and intra-operatively via fluoroscopy. The radio-opaque marker may take many shapes, including but not limited to generally square, rectangular, spherical, oval, ring, and polygonal. Accordingly, the layer regions of the spacer may contain slots or holes to accommodate any number, shape or size of radio-opaque markers. The radio-opaque markers may then be stitched onto the layer regions. Once the radio-opaque marker is securely contained in the slot via embroidery (or any other suitable means), the base textile structure is then folded at the hinge regions such that the layer regions are stacked on top of one another to form the spacer of the implant. The radio-opaque marker may also take the form of a bead that includes a hole through its center. In this way, a stitching thread can pass through the center hole of the radio-opaque bead and secure it into position within the textile spacer.

It will be appreciated that the spacer may incorporate one or more or all of the features described herein and any combination thereof without departing from the scope of the present invention. In fact, layering many individual layer regions, each with different contours, achieves a complex structure that can more accurately conform to an intervertebral disc space. Combining individual layer regions of different contours can create a spacer having the precise anatomical features of an offset dome, a lordosis angle and a taper at the side. With this layering method, an implant can replicate or approximate the exact anatomical shape and structure of an intervertebral disc. This method can then be applied to custom fit an implant to an individual patient in order to better treat his/her pathology.

With each layer region having an orientation in relation to the final shape of the implant, the hinged design of the base textile structure ensures that the layer regions are correctly aligned when stacked on top of one another. By having the layer regions hinged together, the correct combination, order, and orientation of the components will occur every time. This enables consistency and reproducibility in the manufacturing process of the spacer.

The encapsulating jacket may comprise two outer caps (i.e., one outer cap for the top of the implant and one outer cap for the bottom of the implant) and a circumferential lateral barrier disposed between the two outer caps with an attachment flange. The outer caps of the encapsulating jacket may be designed to allow for expansion in order to accommodate any and all shapes of the spacer described herein. By way of example only, the outer cap may have a yarn path that is in a zigzag pattern, similar to that shown and described in above-referenced PCT Application No. PCT/US2008/05254. The zigzag pattern creates three dimensional outer caps to cover the top and bottom of the spacer.

The circumferential barrier of the encapsulating jacket may be comprised of three circumferential layers: an inner layer, a middle layer with an attachment flange, and an outer layer, according to one embodiment of the present invention. The three circumferential layers of the circumferential barrier may have longitudinal load-bearing threads and vertical load-bearing threads. By consisting of longitudinal and vertical load-bearing threads, the three circumferential layers are designed to radially contain the spacer, while sustaining the axial pressure from the adjacent vertebral bodies. By consisting of vertical load-bearing threads, the circumferential barrier holds the layer regions of the spacer together. In this way, the circumferential barrier retains the core shape of the spacer and reinforces the structure of the implant, thereby restoring the height of the intervertebral disc space.

According to the preferred embodiment for the circumferential barrier of the present invention, the inner circumferential layer primarily contains vertical load-bearing threads in order to hold the layer regions of the spacer tightly together. To hold the spacer together, the top layer region of the spacer is stitched to the top edge of the inner circumferential layer and the bottom layer region of the spacer is stitched to the bottom edge of the inner circumferential layer. The middle circumferential layer primarily contains longitudinal load bearing threads to radially contain the spacer. The outer circumferential layer primarily contains both longitudinal and vertical load bearing threads to provide both axial support and to secure attachment for the top and bottom layers of the spacer. It will be appreciated that the number of circumferential layers of the circumferential barrier may be changed without departing from the scope of the present invention. In all instances, the circumferential barrier will retain the height, shape and structure of the implant.

By way of example only, the middle circumferential layer may include an attachment flange having apertures formed therein. The flange extends from the middle layer such that it can be used for affixing the encapsulating jacket to an adjacent anatomic structure (e.g. a vertebral body) to maintain the implant in position before the embroidery becomes encapsulated with scar tissue. Apertures may be optionally provided in the flange to accommodate anchors, such as screws or any other suitable affixation elements (e.g. nails, staples, bone anchors, etc.), which secure the implant to the adjacent spinal vertebrae. Although the encapsulating jacket is shown as having only one attachment flange, it will be appreciated that this number is set forth by way of example only and that the number of flanges may be increased or decreased without departing from the scope of the present invention. Furthermore, it will be appreciated that the attachment flange is not limited to the middle circumferential layer and may be attached to any component of the circumferential barrier and/or encapsulating jacket without departing from the scope of the present invention.

In addition, reinforced stitching may be added throughout the implant in order to prevent anterior-posterior and/or medial-lateral bulging under pressure. The reinforced stitching may be constructed from the same material as the implant, or may be made from different materials, including but not limited to polyester, metal wire, or fiber wire. Using a separate material (e.g. metal fibers) to construct the reinforced stitching may serve dual purposes of preventing midline bulging and acting as a radio-opaque marker.

Several embodiments of assemblies and techniques for inserting an implant into an intervertebral disc space are contemplated according to the present invention. According to a first example of an inserter assembly, the inserter may include a distal engagement region and an elongated handling member. The inserter (of this and subsequent examples) may be composed of any material suitable for inserting an implant into an intervertebral disc space, including but not limited to metal (e.g. titanium, stainless steel, etc.), ceramic, and/or polymer compositions. The distal engagement region of the inserter may be comprised of an insertion plate. The insertion plate has a generally planar rectangular shape, but may take the form of any geometric shape necessary to interact with the implant, including but not limited to generally oval, square, and triangular. The handling member is generally cylindrical in shape. The handling member allows a clinician to manipulate the tool during an implant insertion procedure.

In order to facilitate engagement with the inserter, the implant may include a pocket. The insertion plate engages with the implant by sliding into the pocket. Although slideable engagement is described herein, any suitable means of engagement may be used to engage the insertion plate with the implant, including but not limited to a threaded engagement, snap engagement, hooks, and/or compressive force. Once the insertion plate is disposed within the pocket of the implant, the inserter releasably maintains the implant in the proper orientation for insertion. The implant may then be introduced into an intervertebral disc space while engaged with the inserter and thereafter released.

According to a second example of an inserter assembly, the inserter may include a distal engagement region comprising a pair of prongs and an elongated handling member. Preferably, the insertion prongs are generally cylindrical in shape having a generally circular cross-section, but may take the form of any geometric shape necessary to interact with the implant, including but not limited to generally rectangular, triangular, semi-circular, and the like. In order to facilitate engagement with the insertion prongs, the implant is provided with attached side pockets. Once the insertion prongs engage the implant (e.g. by sliding into the side pockets), the inserter assembly of this example functions in the same manner as the inserter assembly of the first example described above.

According to a third example of an inserter assembly, the inserter may include a pair of elongated handling members and a distal engagement region consisting of a clamping mechanism. The handling members are generally cylindrical in shape and allow a clinician to manipulate the tool during an implant insertion procedure. The clamping mechanism may be comprised of a pair of clamping plates, each having a curved “C”-shaped groove at its distal end. The clamping plates are generally planar rectangular in shape and parallel to one another. The grooved ends of the clamping plates are oriented such that each respective “C” shape faces one another, thereby forming a cradle for engagement with the implant. Preferably, the grooved ends grasp a wireframe disposed within the implant.

In order to provide an attachment point for grasping by an inserter, the implant may contain a wireframe that is sufficiently large and rigid. The wireframe of the implant may be composed of any material suitable for engagement with an inserter, including but not limited to metal (e.g. titanium, stainless steel, etc.), ceramic, and/or polymer compositions. If the wireframe is composed of metal fibers or any other type of radio-opaque material, it may also serve as a radio-opaque marker. The wireframe is a cylindrical frame that runs along the inside perimeter of the rectangular implant and extends past one side of the implant to provide a rail for the inserter to grab onto. Furthermore, the wireframe adds stiffness to the implant to help facilitate insertion. The grooved ends of the clamping plates of the inserter engage with the implant by clamping onto the rail of the wireframe. Once the rail of the wireframe is clamped within the cradle of the inserter, the inserter releasably maintains the implant in proper orientation for insertion. The implant may then be introduced into an intervertebral disc space while engaged with the inserter and thereafter released.

According to a fourth example of an inserter assembly, the inserter may include an elongated handling member and a distal engagement region comprised of a generally cylindrical-shaped threaded engagement feature. The handling member is generally cylindrical in shape and allows a clinician to manipulate the tool during an implant insertion procedure.

In order to facilitate engagement with the inserter, the implant includes an aperture at the proximal side of the implant. The implant may has an engagement plate at the distal side of the implant. The aperture extends inwardly through the implant from the proximal side in a generally perpendicular fashion until it reaches the engagement plate at the distal side of the implant. Preferably, the aperture has a diameter larger than the inserter (if only slightly) such that the inserter may slidably engage with the implant and fit within the aperture. Although shown as having a generally circular cross-section, it will be appreciated that the aperture may be provided having any number of suitable shapes or cross-sections corresponding to that of the inserter, including but not limited to rectangular or triangular.

The engagement plate may be generally rectangular in shape and composed of metal (e.g. titanium, stainless steel, etc.), ceramic, and/or polymer compositions. If composed of metal fibers or any other type of radio-opaque material, the engagement plate may also serve as a radio-opaque marker. To retain the engagement plate, the implant may include a pocket at the distal side. Alternatively, the engagement plate may be stitched under the outer layer or encapsulating jacket of the implant at its distal side. The engagement plate includes at its center a threaded hole to provide a point of attachment for the engagement feature of the inserter. The threaded hole on the engagement plate matches the threaded engagement feature on the inserter so that they can be threadably attached to one another. Other methods of creating a gripping surface are contemplated including but not limited to knurling or facets.

The inserter engages with the implant by entering the aperture on the proximal side of the implant and sliding through the aperture until it reaches the distal side of the implant where it threadedly (or otherwise) engages the engagement plate. Once securely engaged, the inserter releasably maintains the implant in proper orientation for insertion. The implant may then be introduced into an intervertebral disc space while engaged with the inserter and thereafter released.

By way of example only, the following embodiments describe an implant that may be inserted from an anterior aspect of the spine. According to a first example, the implant may include screw holes or apertures dimensioned to receive screws or other affixation elements (e.g. nails, staples, etc.). The implant may be inserted in the intervertebral disc space between two adjacent vertebrae of the spine and thereafter secured by bone screws. The screw holes within the implant may be angled (i.e. not perpendicular) relative to the bone surface such that there is an angle of fixation when the screw is drilled into the inferior vertebra to secure the implant in situ. Angled screw holes within the implant are desirable and advantageous when securing the implant inside an intervertebral disc space because they allow for proper affixation of the screws when there is little access to the inferior vertebra due to the vertical alignment of the adjacent vertebrae.

Although described herein largely in terms of affixing the implant to the inferior vertebra, it will be understood that the implant may be attached to the superior vertebra without departing from the scope of the present invention. This may apply to any embodiment of the implant described herein. In all instances, it is understood that whether the implant is affixed to the inferior vertebra or if the implant is affixed to the superior vertebra, the implant will be situated in the intervertebral disc space either way and will result in the repair/reconstruction of the affected area.

According to a second example, the implant may include attachment flanges centrally located on the anterior side of the implant. Each attachment flange may have a screw hole dimensioned to receive screws or other affixation elements (e.g. nails, staples, etc.). The implant is inserted between two bone surfaces of a joint (e.g. an intervertebral disc space between two adjacent vertebrae and/or another joint elsewhere in the body) to prevent bone-on-bone contact. Once the implant is inserted, screws pass through the screw holes in the attachment flanges of the implant. The screws are then drilled into the bone to secure the implant in position.

In a further embodiment of the present invention, the attachment flange may be comprised of multiple layers folded on top of one another (instead of a single layer extending from the implant). In all cases, it will be understood that the attachment flange of the implant results in the implant being secured within the joint, thereby repairing/reconstructing the degenerative joint by preventing bone-on-bone contact and preserving the natural motion of the joint.

According to a third example, the implant may be attached to a fixation buttress. The fixation buttress may be made from (for example, including but not limited to) metal (e.g. titanium), ceramic, plastic, and/or polymer compositions. The fixation buttress may include a screw hole dimensioned to receive screws or other affixation elements (e.g. nails, staples, etc.) for securing the implant to bone.

According to a fourth example, the implant may have pockets in order to facilitate clip-on fixation buttresses. By way of example only, the pockets on the implant may be extra layers of embroidered fabric attached to the side of the implant with openings for engagement with the clips of the clip-on flanges. The clip-on flanges may be composed of any suitable material, including but not limited to metal (e.g. titanium), ceramic, plastic, or polymer compositions.

It will be appreciated that the implant may incorporate one or more or all of the features described herein and any combination thereof without departing from the scope of the invention. It will also be appreciated that the features described above can be applied to any of the embodiments disclosed herein. While this invention has been described in terms of a best mode for achieving the developers' objectives, it will be understood by those skilled in the art that variations may be accomplished in view of these teachings without deviating from the spirit or scope of the invention.

### BRIEF DESCRIPTION OF THE DRAWINGS

Many advantages of the present invention will be apparent to those skilled in the art with a reading of this specification in conjunction with the attached drawings, wherein like reference numerals are applied to like elements and wherein:

FIG. 1 is a perspective view of an example of a textile-based spinal implant dimensioned for lateral insertion according to a first embodiment of the present invention;

FIG. 2 is a perspective view of a spacer forming part of the implant of FIG. 1;

FIG. 3 is a top plan view of a first example of a base textile structure used to form the spacer of FIG. 2;

FIG. 4 is a perspective view of the base textile structure of FIG. 3 being folded to form the spacer, which is then placed within an encapsulating jacket forming part of the of the implant of FIG. 1;

FIGS. 5-6 are perspective views of second and third examples, respectively, of a base textile structure used to form the spacer of FIG. 2;

FIGS. 7-8 are side cross-sectional views of the base textile structure of FIG. 3 being folded to form the spacer of FIG. 2;

FIG. 9 is a side cross-sectional view of the spacer of FIG. 8 during a pre-settling process according to one embodiment of the present invention;

FIGS. 10-11 are side cross-sectional views of an implant containing the pre-settled spacer of FIG. 9 positioned within an intervertebral disc space;

FIGS. 12-13 are top plan and side cross-sectional views, respectively, of a non-contoured single layer region forming part of the spacer of FIG. 2;

FIG. 14 is a side cross-sectional view of a spacer with an overall tapered shape according to one embodiment of the present invention;

FIG. 15 is a top plan view of a first example of an optional contoured single layer region forming part of the spacer of FIG. 2;

FIGS. 16-17 are top plan and side cross-sectional views, respectively, of a second example of an optional contoured single layer region forming part of the spacer of FIG. 2;

FIGS. 18-19 are top plan and side cross-sectional views, respectively, of a third example of an optional contoured single layer region forming part of the spacer of FIG. 2;

FIGS. 20-21 are top plan views of fourth and fifth examples, respectively, of an optional contoured single layer region forming part of the spacer of FIG. 2;

FIG. 21 is a top plan view of a fifth example of an optional contoured single layer region forming part of the spacer of FIG. 2;

FIG. 22 is a top plan view illustrating sequential layering in the formation of the contoured single layer region of FIG. 20;

FIG. 23 is a top plan view of a sixth example of an optional contoured single layer region forming part of the spacer of FIG. 2;

FIGS. 24-25 are top plan and side cross-sectional views, respectively, of a seventh example of an optional contoured single layer region forming part of the spacer of FIG. 2;

FIGS. 26-27 are top plan and side cross-sectional views, respectively, of an eighth example of an optional contoured single layer region forming part of the spacer of FIG. 2;

FIG. 28 is a perspective view of the contoured layer region of FIG. 26 coupled together via supplemental stitches;

FIGS. 29-31 are top plan views of ninth, tenth, and eleventh examples, respectively, of an optional contoured single layer region forming part of the spacer of FIG. 2;

FIGS. 32-33 are perspective and partial side cross-sectional views, respectively, of a tapered textile-based implant according to one embodiment of the present invention;

FIG. 34 is a top plan view of a base textile structure used to form a tapered textile-based core forming part of the tapered textile-based implant of FIG. 32;

FIGS. 35-37 are cross-sectional views of examples of various folding techniques for folding the base textile structure of FIG. 34;

FIGS. 38-39 are cross-sectional views of examples of tapered textile-based cores according to the present invention;

FIG. 40 is top plan view of an example of a base textile structure used to form a tapered textile-based core according to one embodiment of the present invention;

FIG. 41 is a cross-sectional view of a of a tapered textile-based core created by folding the base textile structure of FIG. 40;

FIG. 42 is a top plan view of another example of a base textile structure used to form a tapered textile-based core according to present invention;

FIG. 43 is a cross-sectional view of a of a tapered textile-based core created by folding the base textile structure of FIG. 42;

FIG. 44 is a top plan view of layer region having three different stitching orientations used to create three sections of varying stitch density on a single layer region according one embodiment of the present invention;

FIG. 45 is a top plan view of an example of a base textile structure used to form a tapered textile-based core, formed using several layer regions of FIG. 44;

FIG. 46 is a cross-sectional view of a tapered textile-based core created by folding the base textile structure of FIG. 45;

FIG. 47 is a top plan view of a layer region having three different stitching orientations used to create five sections of varying stitch density on a single layer region according to a further embodiment of the present invention;

FIG. 48 is a top plan view of an example of a base textile structure used to form a tapered textile-based core, formed using several layer regions of FIG. 47;

FIG. 49 is a cross-sectional view of a tapered textile-based core created by folding the base textile structure of FIG. 48;

FIG. 50 is a top plan view of an example of a base textile structure used to form a tapered textile-based core having four layer regions each with three sections of varying yarn spacing according to another embodiment of the present invention;

FIG. 51 is a cross-sectional view of a tapered textile-based core created by folding the base textile structure of FIG. 50;

FIG. 52 is a top plan view of yet another example of a base textile structure used to form a textile-based core according to the present invention;

FIGS. 53-54 are cross-sectional and perspective views, respectively, of a tapered textile-based core created by folding the base textile structure of FIG. 52;

FIG. 55 is a top plan view of a base textile structure capable of being folded to produce a tapered textile-based core within an encapsulating jacket according one embodiment of the present invention;

FIG. 56 is a top plan view of a base textile structure capable of being folded to create the tapered textile-based core of FIG. 55;

FIGS. 57-58 are perspective views of the spacer of FIG. 2 containing first and second examples, respectively, of an optional internal radio-opaque marker;

FIG. 59 is a top plan view of a single contoured layer region containing a slot to accommodate a radio-opaque marker of FIG. 57;

FIG. 60 is a top plan view of a single contoured layer region containing three slots to accommodate the radio-opaque markers of FIG. 58;

FIGS. 61-63 are perspective views of examples of radio-opaque markers in the form of an elliptical bead, spherical bead, and tubular bead, respectively, according to the present invention;

FIG. 64 is a perspective view of the spacer of FIG. 2 containing a third example of an optional radio-opaque marker;

FIG. 65 is a perspective view of a portion of the spacer of FIG. 64 being folded into an encapsulating jacket according to the present invention;

FIG. 66 is perspective view of a portion of the spacer of FIG. 64, illustrating in particular the placement of the ring marker between adjacent textile layers;

FIGS. 67-69 are cross-sectional views of the portion of FIG. 66, further illustrating the placement of the ring marker between adjacent textile layers;

FIGS. 70-74 are top plan views of a spacer of FIG. 64 illustrating sequential steps in manufacturing the layer retaining the ring marker according to the present invention;

FIG. 75 is a top plan view of one example of a base textile structure including contoured layer regions and a slot to accommodate a radio-opaque marker to form an contoured version of the spacer of FIG. 57;

FIG. 76 is a top plan view of an example of a base textile structure including contoured layer regions and a radio-opaque marker of FIG. 64;

FIG. 77 is a perspective view of several contoured single layer regions of FIG. 26 coupled together at lateral hinge regions to form an optional contoured spacer;

FIG. 78 is a perspective view of a spacer having an overall tapered and dome shape according to one embodiment of the present invention;

FIGS. 79-80 are side cross-sectional views taken at planes A and B, respectively, of the spacer of FIG. 78;

FIG. 81 is a perspective view of an example of an encapsulating jacket forming part of the implant of FIG. 1;

FIG. 82 is a top plan view of an outer cap used to form the top and bottom of the encapsulating jacket of FIG. 81;

FIG. 83 is a top view of a circumferential barrier forming part of the encapsulating jacket of FIG. 81;

FIGS. 84-86 are plan views of inner, middle, and outer circumferential layers, respectively, forming parts of the circumferential barrier of FIG. 83;

FIGS. 87-89 are top cross-sectional, perspective, and side views, respectively, of an example of a textile-based implant containing reinforced stitching, according to one embodiment of the present invention;

FIGS. 90-91 are top views of an example of a textile-based implant including a single pocket and an inserter assembly for inserting the implant into an intervertebral space according to the present invention;

FIG. 92-93 are top views of an example of a textile-based implant including a pair of side pockets and an inserter assembly for inserting the implant into an intervertebral space according to the present invention;

FIG. 94-95 are top views of an example of a textile-based implant including a wire frame and an inserter assembly for inserting the implant into an intervertebral space according to the present invention;

FIG. 96 is a perspective view of an example of a textile-based implant including an engagement plate and an inserter assembly for inserting the implant into an intervertebral space according to the present invention;

FIGS. 97-98 are plan and sectional views, respectively, of the engagement plate of FIG. 96;

FIGS. 99-101 are top, perspective, and side sectional views, respectively, of a textile-based spinal implant dimensioned for anterior insertion according to a second embodiment of the present invention;

FIG. 102 is a perspective view of the implant of FIG. 99 positioned within a intervertebral disc space in the spine;

FIGS. 103-104 are top and partial cross-sectional views, respectively, of an example of the textile-based implant of FIG. 99 including three optional attachment flanges;

FIG. 105 is a partial side cross-sectional view of the implant of FIG. 103 including a screw and showing the angle of fixation between the screw and the inferior vertebra;

FIG. 106-107 are top and partial cross-sectional views, respectively, of an example of the textile-based implant of FIG. 99 including an attachment flange with three screw holes;

FIG. 108 is a partial side cross-sectional view of the implant of FIG. 106 including a screw and showing the angle of fixation between the screw and the inferior vertebra;

FIG. 109 is a side cross-sectional view of part of the implant of FIG. 99 having an optional folded attachment flange;

FIG. 110 is a partial side cross-sectional view of a textile-based implant and attached fixation buttress according to one embodiment of the present invention;

FIG. 111 is a perspective view of the fixation buttress of FIG. 110;

FIG. 112 is a perspective view of a textile-based implant including pockets and clip-on fixation buttresses, according to one embodiment of the present invention; and

FIG. 113 is a perspective view of the clip-on fixation buttress of FIG. 112.

### DESCRIPTION OF THE PREFERRED EMBODIMENT

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure. The systems disclosed herein boast a variety of inventive features and components that warrant patent protection, both individually and in combination.

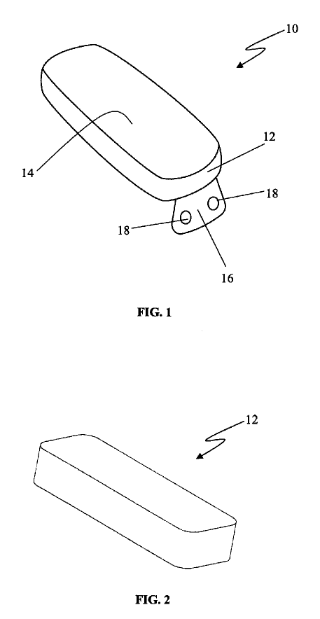

FIG. 1 illustrates an example of a textile-based surgical implant 10 according to one embodiment of the present invention. Implant 10 includes a spacer 12 (shown by itself in FIG. 2) disposed within an encapsulating jacket 14. The implant 10 may be inserted through any number of suitable surgical approaches, including but not limited to lateral, anterior, anterior-lateral, postero-lateral, and/or posterior approaches. When applied to spine surgery and inserted into an intervertebral disc space, the implant 10 restores the normal height of the intervertebral disc space, while advantageously preserving the natural motion of the spine. The textile construction of the implant 10 is beneficial because it is generally compliant and thereby restores and/or improves spinal motion. The compliant nature of the textile-based implant 10 provides the required flexibility and elasticity to advantageously support physiological movements, as opposed to fusion surgery which forms a boney bridge between adjacent vertebral bodies. In addition, as will be described in greater detail below, the porosity and biocompatibility of the textile-based implant 10 facilitates tissue ingrowth throughout part or all of the implant, which helps to secure and encapsulate the implant 10 in the intervertebral space.

The jacket 14 may be constructed from any of a variety of textile materials (e.g. polyester fiber, polyethylene (including ultra-high molecular weigh polyethylene), polyclycolic acid, polylactic acid, etc.) via any number of textile processing techniques (e.g. embroidery, weaving, three-dimensional weaving, knitting, three-dimensional knitting, injection molding, compression molding, cutting woven or knitted fabrics, etc.). The jacket 14 may encapsulate the spacer 12 fully (i.e. disposed about all surfaces of the spacer 12) or partially (i.e. with one or more apertures formed in the jacket 14 allowing direct access to the spacer 12). The various layers and/or components of the spacer 12 may be attached or unattached to the encapsulating jacket 14. The jacket 14 may optionally include one or more fixation elements for retaining the jacket 14 in position after implantation, including but to limited to at least one flange 16 extending from or otherwise coupled to the jacket 14 and screws or other affixation elements (e.g. nails, staples, sutures, adhesives, tacks, etc.) to secure the flange 16 to an adjacent anatomical structure (e.g. vertebral body). This may be facilitated by providing one or more apertures 18 within the flange 16 dimensioned to receive the screws or other fixation elements.

Although generally described herein as a two-part construct (with a spacer 12 within an encapsulating jacket 14) it will be appreciated that the implant 10 may include a spacer 12 without the encapsulating jacket 14, wherein the shape of the spacer 12 is maintained through any number of suitable techniques, including but not limited to stitching the spacer 12 together via supplemental stitches (not shown). It will also be appreciated that the implant 10 may be comprised of a spacer 12 and an encapsulating jacket 14 made from a single continuous embroidery. In all instances described herein throughout, the implant 10 provides the necessary height and shape to restore the natural curvature of the spine, along with the required flexibility and elasticity to partially or fully restore or improve the physiologic movements of the spine.

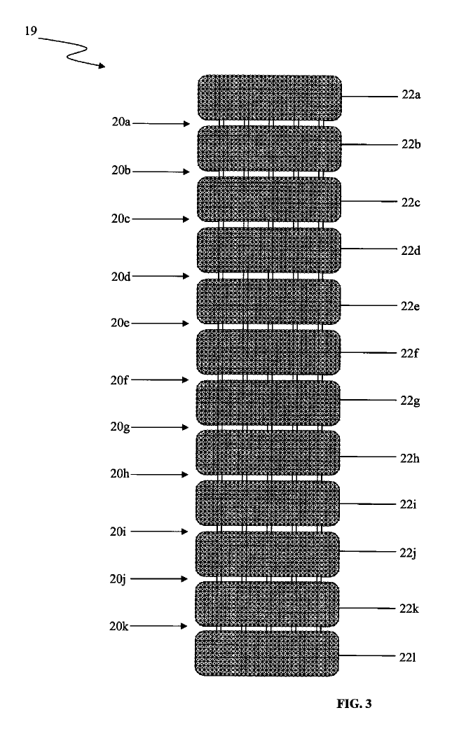

A variety of embodiments may be used in constructing the implant 10 of the present invention. FIGS. 3-4 collectively illustrate a on example of a method for creating an implant 10 according to the present invention. In this method, the first step involves manufacturing a base textile structure 19 used to form the spacer 12. The base textile structure 19 is preferably manufactured via an embroidery process using any number of biocompatible filament materials (including but not limited to polyester thread). This embroidery process may or may not include a backing layer. For example, stitches may be added onto a flat fabric such as a backing layer or base cloth. Subsequently, the backing layer may be dissolved. It will be appreciated that the base textile structure 19 may be formed from different materials and/or made from techniques other than embroidery (e.g. weaving, etc.) without departing from the scope of the present invention.

By way of example only, base textile structure 19 is comprised of a plurality of hinged embroidered layer regions 22a-22l. The layer regions 22a-22l of the base textile structure 19 are connected together and separated by a distance to form a plurality of hinge regions 20a-20k between the layer regions 22a-22l, respectively. The base textile structure 19 is then folded to form the spacer 12 and placed within the encapsulating jacket 14, as shown in FIG. 4. In this example, the layer regions 22a-22l are folded at the hinge regions 20a-20k such that the layer regions 22a-22l are stacked consecutively on top of one another in a back-and-forth or accordion-like manner (i.e., with layer region 22k folded on top of layer region 22l, layer region 22j folded on top of layer region 22k, layer region 22i folded on top of layer region 22j, layer region 22h folded on top of layer region 22i, layer region 22g folded on top of layer region 22h, layer region 22f folded on top of layer region 22g, layer region 22e folded on top of layer region 22f, layer region 22d folded on top of layer region 22e, layer region 22c folded on top of layer region 22d, layer region 22b folded on top of layer region 22c, and layer region 22a folded on top of layer region 22b.)

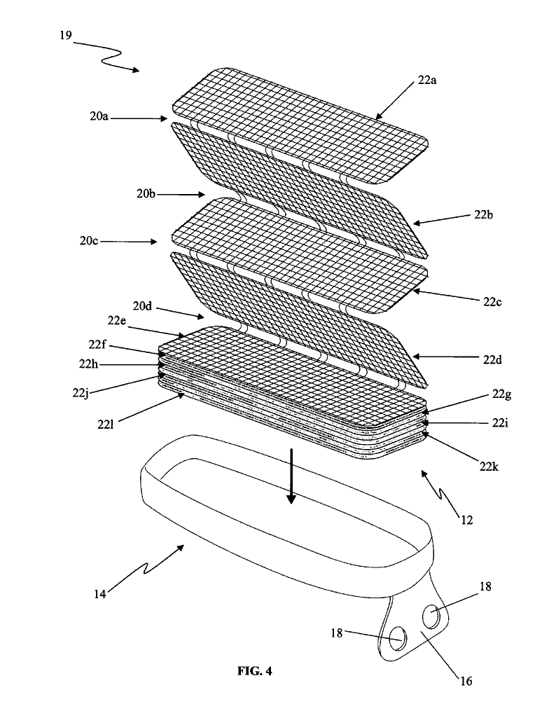

Although the folding technique of the layer regions 22a-22l is shown and described herein by example as a back-and-forth or accordion-like manner, it will be appreciated that the folding process may be performed in any number of manners without departing from the scope of the present invention. It will also be understood that the placement of the hinges between layer regions 22a-22l may vary without departing from the scope of the present invention. For example, FIGS. 3-4 depict the layer regions 22a-22l having hinge regions 20a-20k disposed on the long edges of the rectangular-shaped layer regions. Another example is shown in FIG. 5, in which the layer regions 24a-24c may have hinge regions 26a-26b disposed on the short edges 28 of the rectangular-shaped layer regions 24a-24c (as opposed to having hinge regions disposed on the long edges 30).

The inclusion of hinged layer regions (e.g. 22a-22l in FIGS. 3-4 and 24a-24c in FIG. 5) can be beneficial to the manufacturing of a spacer 12. With the hinge regions (e.g. 20a-20k in FIGS. 3-4 and 26a-26b in FIG. 5), perfect alignment, stability, and accuracy may be attained when folding the layer regions on top of one another to form the spacer 12. When the folding process occurs, alignment of the layer regions is relatively easy because of the inter-connectivity of the layer regions via the hinge regions. Placement of the layer regions on top of one another is guided and advantageously constrained by the distance/length of the hinge regions. Having this defined space between the connected layer regions facilitates the accurate and consistent placement of the layer regions on top of one another to form a stack. As a result, the spacer 12 that is formed after folding has a uniform overall shape.

Hence, the hinged design allows for reproducibility and acts as a time saving feature. Since the hinge regions assist in the folding of the layer regions to form the spacer 12, efficiency is increased during the manufacture of one spacer and in mass production. In addition, the spacer 12 has more stability because the layer regions are connected, as opposed to a stack of layer regions that are not connected to each other. Having hinged layer regions helps secure the overall structure of the stacked layer regions and ensure that the spacer 12 stays together in one piece. Furthermore, the hinged design allows for the manufacture of any quantity of virtually identical implants 10, thus the implants are easy to reproduce.

Although not shown in FIGS. 3-4, it is also contemplated that the layer regions 22a-22l may be coupled together without the use of hinge regions, for example, via the use of other fixation or coupling mechanisms, including but not limited to supplemental stitching and/or adhesives capable of coupling adjacent or multiple layer regions together. It will be also appreciated that although shown and described by way of example only as having hinge regions 20a-20k, the spacer 12 may have no hinges and may be comprised of a stack of individual layer regions 22a-22l that are not connected, or the base textile structure 19 may be a single continuous sheet of embroidery that is folded to form a plurality of layers stacked on top of one another to form the spacer 12. In all instances, the implant 10 restores the height of the intervertebral disc space, while advantageously preserving the natural motion of the spine. Moreover, although shown and described herein as having twelve layer regions 22a-22l and eleven hinge regions 20a-20k, the actual number of layer regions and hinge regions may be increased or decreased without departing from the scope of the present invention. This may be done for any number of different purposes, including but not limited to varying the height of the spacer 12.

By way of example only, FIG. 6 illustrates an example of a spacer 12a having no hinges and comprised of a stack of individual layer regions 32a-32c that are not connected through hinge regions. According to this example, having individual stacked layer regions 32a-32c without hinges may present a manufacturing advantage in that a manufacturing defect affecting a single layer (e.g. thread break for a single layer region) would result in the rejection of only that defective layer region. In comparison, with the embodiments where the layer regions are coupled together via hinges or comprised of a single continuous sheet, a single fault within any part of the construction (e.g. a thread within a single hinge region) would result in the entire implant being rejected.

After the layer regions 32a-32c are stacked consecutively on top of one another, they are coupled together via supplemental stitches 34 to form the spacer 12a. More specifically, layer region 32a is placed on top of layer region 32b, and layer regions 32a and 32b are then placed on top of layer region 32c to form a stack. After forming a stack of individual layer regions 32a-32c to create the spacer 12a, supplemental stitches 34 are placed between layer regions 32a and 32b as well as between layer regions 32b and 32c. Supplemental stitches 34 hold the layer regions 32a-32c together and maintain the overall shape of the spacer 12a. It will be appreciated that the number and placement of the supplemental stitches 34 is set forth by way of example only, and may be varied in order to couple adjacent or multiple layer regions together. It will also be understood that the number of layer regions 32a-32c is set forth by way of example only to illustrate the formation of a spacer 12a through individual stacked layer regions, and that in practice any number of layer regions may be used to accommodate the needs of a user.

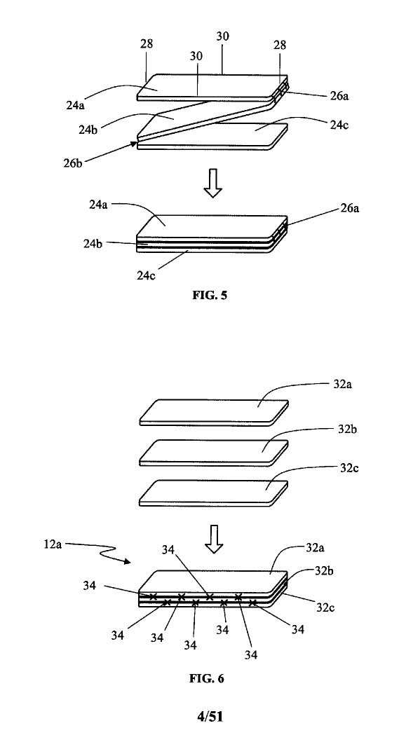

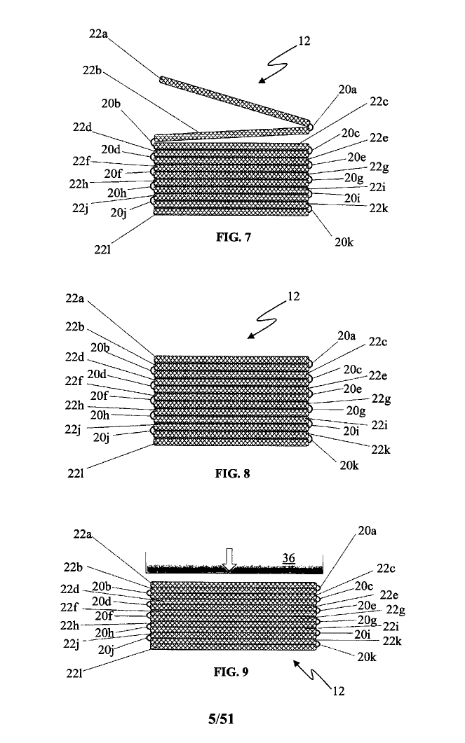

After the various layer regions of the spacer 12 are folded (or otherwise arranged) but before implantation, the stack of layer regions that comprise the spacer 12 may be pre-settled as shown in FIGS. 7-9. This process is similar to the pre-settling process shown and described in commonly owned and co-pending PCT Application Serial No. PCT/US2008/053315, referenced above. This process is explained herein using the example of spacer 12 from FIGS. 3-4. During the pre-settling process, as shown by example in FIG. 9, the stack of layer regions 22a-22l are compressed under a high load 36 in order to make the implant 10 more dimensionally stable and compact. Once implanted and used to restore the intervertebral disc height, the pre-settled stack of layer regions 22a-22l that comprise the spacer 12 will be more likely to keep its shape while sustaining the axial load and pressure from the adjacent vertebrae. This process further ensures that a properly sized implant 10 may be selected prior to surgery, and also that introduction of the implant will be easier since an unsettled implant must be larger to accommodate the natural settling that generally occurs after implantation. The spacer 12 may be pre-settled before or after placement into the encapsulating jacket 14.

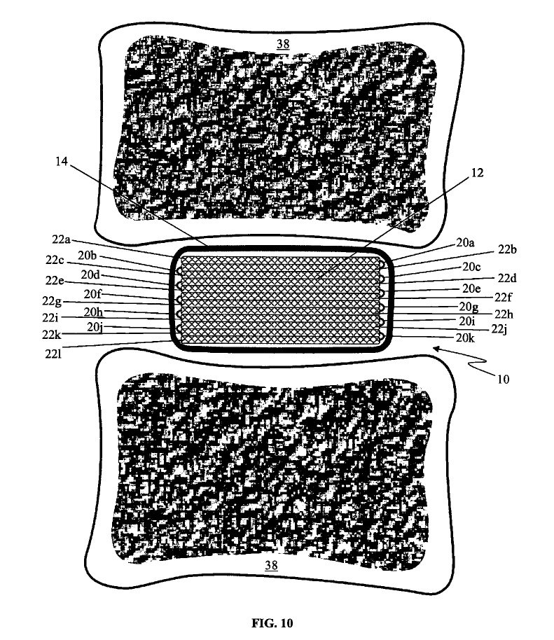

FIGS. 10 & 11 illustrate the benefits of having a stack of layer regions 22a-22l to comprise the structure of the spacer 12, as opposed to a rigid block used for fusion. The stack of layer regions 22a-22l advantageously allows for natural motion, providing the ability to accommodate bending movements while simultaneously preserving the overall disc height. The stack of layer regions 22a-22l compresses and fans out according to the pressures to which it is subjected. For example in FIG. 10, when the implant 10 is being compressed between the adjacent vertebrae 38, the stack of layer regions 22a-22l of the spacer 12 is very stable and offers resistance to the compounding load, thus maintaining the disc height. As shown in FIG. 11, when the implant 10 undergoes flexion, the stack of layer regions 22a-22l can react with some flexibility yet still maintain the disc height.

During bending, the individual layer regions 22a-22l can expand by fanning-out or “opening out” slightly. The hinge regions 20a-20k help facilitate bending movements by holding the layer regions together and, at the same time, allowing for the accordion-like opening between the layer regions as the implant 10 is flexed. Consequently, the implant 10 conforms to the shape of the intervertebral space into which it is inserted because of the potential for the layer regions 22a-22l to be able to separate slightly yet maintain proper positioning. By allowing mobility while preventing overall height loss, the stack of layer regions 22a-22l that comprise the spacer 12 assists in preserving the natural motion of the spine.

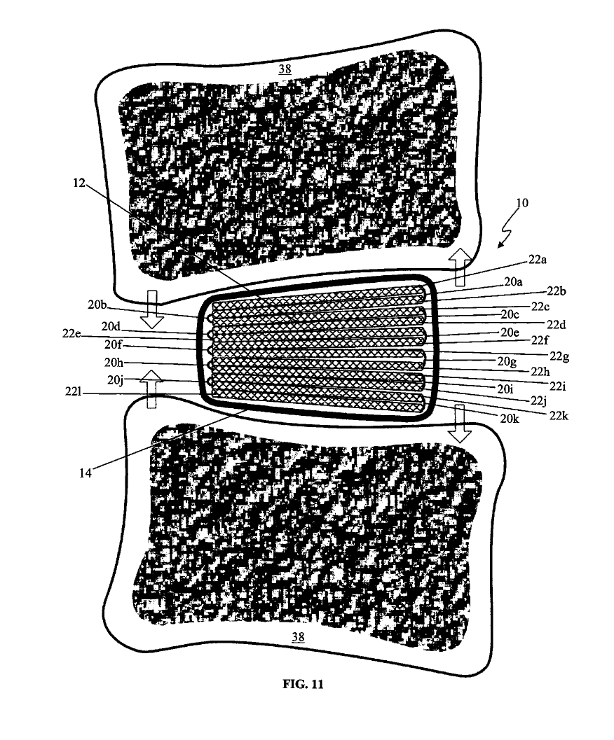

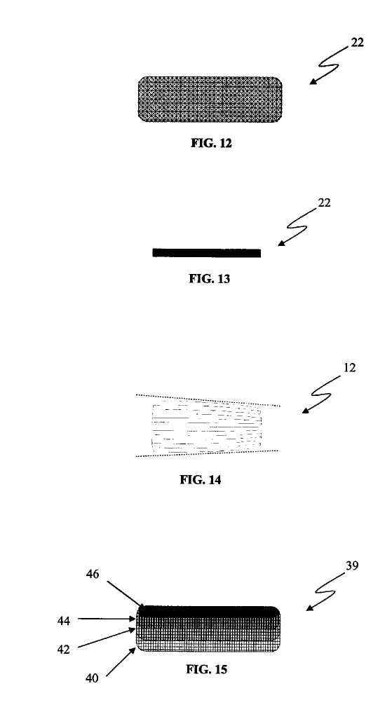

FIG. 12 illustrates a plain layer region 22 used to form a spacer 12. FIG. 13 illustrates the flat cross-section of a plain layer region 22. Plain layer regions 22 may be used to build the height of a spacer 12 and also to impart a smooth surface for the top and bottom layers.

Although shown in FIGS. 1-11 and further as having a generally rectangular shape, the spacer 12 may be provided in any number of suitable dimensions depending upon the surgical application and patient pathology. The spacer 12 may have many shapes, for example generally square, rectangular, spherical, wedge, oval, elliptical, trapezoidal, and polygonal shape. The spacer 12 may have a generally flat upper surface and a generally flat lower surface, or one or both surfaces may be convex or concave. One of the upper and lower surfaces of the spacer 12 may be convex and the other surface may be concave.

While the spacer 12 as described thus far has a shape specifically dimensioned for lateral insertion (i.e. insertion from the side of a patient) of the implant 10 using a minimally invasive surgical technique, the spacer 12 may be provided having any number of different shapes to facilitate insertion via any number of surgical approaches, including anterior (shown below), antero-lateral, postero-lateral and posterior surgical approaches to the spine. In all instances, the implant 10 restores the normal height of the intervertebral disc space, while advantageously preserving the natural (or approximately natural) motion of the spine. In addition, vertically oriented holes or apertures (not shown) running from the top of the spacer 12 to the bottom of the spacer 12 may be included in order to facilitate the spread and nourishment of tissue ingrowth.

A variety of features may be incorporated into the spacer 12 to match (or approximate) the natural curvature of the spine and thereby support the full range of physiological movements. For example, each single layer region 22 of the spacer 12 may be contoured such that when the individual layer regions are folded and stacked on top of one another (as described in detail above) the spacer 12 achieves an anatomical dome shape for the top and bottom of the implant 10. By doing so, the implant 10 would mimic the anatomical shape of an intervertebral disc. In addition, each single layer region 22 of the spacer 12 may be contoured to achieve an overall tapered shape, as shown in the cross-sectional view of the spacer 12 in FIG. 14 and described in further detail below. Thus, when the individual layer regions are folded and stacked on top of one another, the overall shape of the spacer 12 matches the natural lordotic and/or kyphotic angles in any given region of the spine (i.e. lordosis in the cervical and lumbar regions of the spine and kyphosis in the thoracic region of the spine.)

This means that the spacer 12 may have a cross-sectional shape in which the vertebral-contacting surfaces are not parallel. This includes (but is not limited to) cross-sectional shapes wherein the vertebral-contacting surfaces are partially angled relative to one another (e.g. along a portion of the cross-section), fully angled relative to one another (e.g. along all or a substantial portion of the cross-section), generally angled relative to one another (e.g. one or more generally flat regions arranged in a decreasing or increasing “stair step” fashion), curved (e.g. partially, fully, generally, and/or slightly) relative to one another, and/or any combination of the above. In all instances, the implant 10 restores the normal height of the intervertebral disc space, while advantageously preserving the natural motion of the spine.

As described above, the individual layer regions of the spacer may be optionally contoured or generally tapered in different ways to replicate the anatomical shape of an intervertebral disc. Thus, for each specific example described herein throughout, if the spacer contains multiple contoured layer regions or general tapering of the several examples described below, then the spacer will correspondingly result in a contoured and/or tapered cross-sectional shape as a result of the individual contoured layer regions building the height of the spacer when folded and stacked on top of one another. In this way, the overall shape of the spacer replicates (or approximates) the anatomical shape of an intervertebral disc, thereby matching (or approximating) the natural curvature of the spine.

Moreover, although the specific examples described herein involve a particular number of layers and/or sections and/or elements, it should be appreciated that these specific arrangements are set forth by way of example only and that the number of layers and/or sections and/or elements may be increased or decreased without departing from the scope of the present invention. This may be done for any number of different purposes, including but not limited to varying the height and/or the angle of the single layer region (and, in effect, the spacer), and varying the contoured cross-sectional shape of the single layer region (and, in effect, the spacer) to achieve an anatomical dome shape and/or a tapered shape and/or a shape that is custom fit to suit an individual patient. It will also be appreciated that the shape, size, and/or placement of the various layers and/or sections and/or elements is set forth by way of example only, and may be changed to accommodate a wide variety of surgical requirements, contours and/or to suit an individual patient. Any number of suitable dimensions may be provided depending upon the surgical application and patient pathology. In all instances, it will be understood that the implant restores the normal height of the intervertebral disc space, while advantageously preserving the natural (or approximate natural) motion of the spine.

FIG. 15 illustrates a first example of an optional contoured single layer region 39 of the spacer 12 according to one embodiment of the present invention. In this example, a layer region 39 may be repeatedly overstitched in different sections using thread of the same thickness. As shown by way of example only, repeated overstitching advantageously forms sections of different height, for example a first section 40 comprising the first layer of stitching, a second section 42 comprising a second layer stitched on top of the first layer to form double stitching, a third section 44 comprising a third layer stitched on top of the first and second layers to form triple stitching, and a fourth section 46 comprising a fourth layer stitched on top of the first, second, and third layers to form quadruple stitching. The key is that layer region 39 results in a textile layer having a contoured cross-sectional shape (e.g. tapered) due to the sections of varying height 40-46 that are formed from repeated overstitching. The layer regions 39 also contribute to the stability of the contoured structure of the implant 10 because each layer region 39 reaches to the edge of the spacer 12.

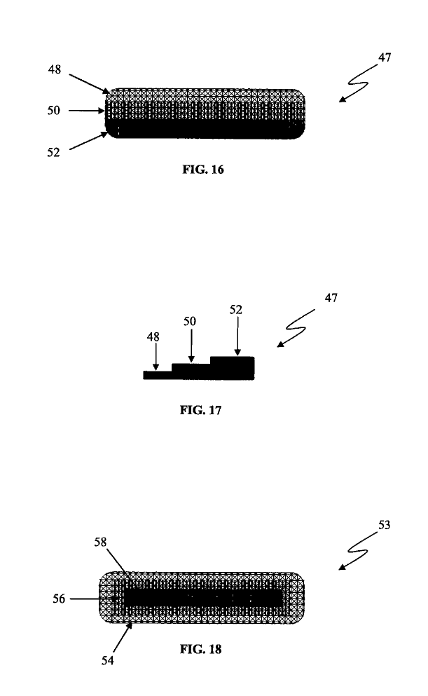

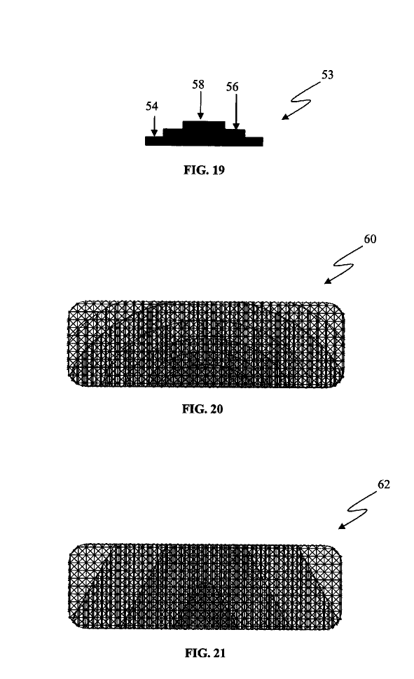

FIGS. 16 & 17 illustrate an example of an optional contoured single layer region 47 having three sections 48-52 of stitching densities used to form a tapered cross-section. By way of example only, FIGS. 18 & 19 illustrate a single layer region 53 having three centrally-aligned sections 54-58 of different sizes and stitching densities used to form a dome shaped cross-section. Due to the different placement, sizes, and densities of sections 54-58, the dome shaped layer region 53 has a convex upper surface with a tapered-off periphery.

By varying the respective and/or relative shapes, sizes, quantity and/or placement of the sections 40-46 in FIG. 15 and/or contours of the individual layer regions 39, the spacer 12 may be dimensioned with varying structures to accommodate a wide variety of surgical requirements. The spacer 12 may be provided in any number of suitable dimensions depending upon the surgical application and patient pathology. The different embodiments of the layer regions 39, 47, 53 containing different contours described herein may be used in combination in order to achieve the final core shape required. For example, as shown in FIGS. 20 & 21, more complex designs can be readily generated (e.g. a semi-circular or triangular design) using various sections of stitching having different shapes and sizes in order to produce custom profiles on a layer region. As shown in the semi-circular design of the single layer region 60 of FIG. 20 and the triangular design of the single layer region 62 of FIG. 21, both layer regions 60, 62 contain a combination of the tapered and dome shaping described above.

As a result of the combined taper and dome shape on the layer regions 60, 62, when multiple layer regions are stacked together to form the spacer 12, precise contours for the spacer may replicate (or approximate) an actual intervertebral disc. More specifically, the angle of natural curvature in the spine (i.e. lordosis in the cervical and lumbar regions of the spine and kyphosis in the thoracic region of the spine) may be matched due to the contoured shape of the spacer 12. In addition, due to the dome shape of the spacer 12, more surface contact may be achieved between the upper and lower surfaces of the implant 10 to the adjacent upper and lower vertebrae. Thus, more tissue ingrowth is facilitated because of the increased surface contact, which may assist in keeping the implant 10 in situ. In all instances, the implant 10 restores the normal height of the intervertebral disc space, while advantageously preserving the natural (or approximately natural) motion of the spine.

FIG. 22 illustrates how one example of a method for adding contour to a single layer region is applied to create the complex semi-circular design of the single layer region 60 in FIG. 20 having a tapered and dome shape. According to this method, in order to achieve the end result of layer region 60, four sections 62-68 of different sizes and stitching densities are used. Semi-circular shaped sections 64-68 are repeatedly overstitched onto base section 62 (which is similar to plain layer region 22 in FIG. 5A) using thread of the same thickness. As shown by way of example only, repeated overstitching advantageously forms sections of different height, for example a first section 62 comprising the first layer of stitching, a second section 64 comprising a second layer stitched on top of the first layer to form double stitching, a third section 66 comprising a third layer stitched on top of the first and second layers to form triple stitching, and a fourth section 68 comprising a fourth layer stitched on top of the first, second, and third layers to form quadruple stitching. The key is that layer region 60 results in a textile layer having a contoured cross-sectional shape that is semi-circular, tapered, and dome-shaped due to the sections 62-68 of varying height and size that are formed from repeated overstitching.

Furthermore, FIG. 22 illustrates how additional sections of varying density may be created by alternating the stitch paths in sections 64-68. For example, alternate double stitch paths 70 are omitted in region 72 of section 64; alternate double stitch paths 76 are omitted in region 78 of section 66; and alternate double stitch paths 80 are omitted in region 82 of section 68. As a result of omitting alternate double stitch paths 70, 76, 80 in regions 72, 78, 82 of sections 64-68, three new sections of different densities 72, 78, 82 are formed. Accordingly, layer region 60 has seven sections 62, 64, 66, 68, 72, 78, 82 of different sizes and stitching densities in total. Increasing the number of sections of varying stitching density reduces the height differential between the adjacent sections. This, in turn, causes the layer region 60 to have a tapered shape with a smoother gradient profile. Due to the semi-circular design and alignment of each of the sections 64, 66, 68, 72, 78, 82, a dome shape is created starting from the bottom center of the layer region 60 and tapering off at the periphery. The cumulative effect of having sections 62-68 in addition to sections 72, 78, 82 (created by alternating the stitch paths) is a smooth gradation of textile thickness from bottom center to the top corners of the layer region 60.

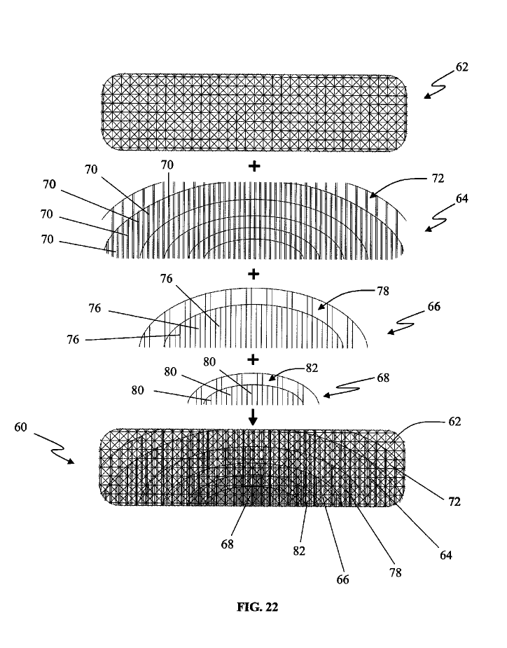

FIG. 23 illustrates another example of an optional contoured single layer region 84 of the spacer 12 according to another embodiment of the present invention. In this example, threads of varying thickness or diameter are used to form sections having different height. As shown by way of example only, the biocompatible filament material includes a section of relatively large diameter thread 86, a section of relatively small diameter thread 88, and a section of intermediate diameter thread 90. The key is that layer region 84 results in a textile layer having a contoured cross-sectional shape (e.g. tapered) due to the sections of varying height 86-90 that are formed from the different thread thicknesses/diameters.

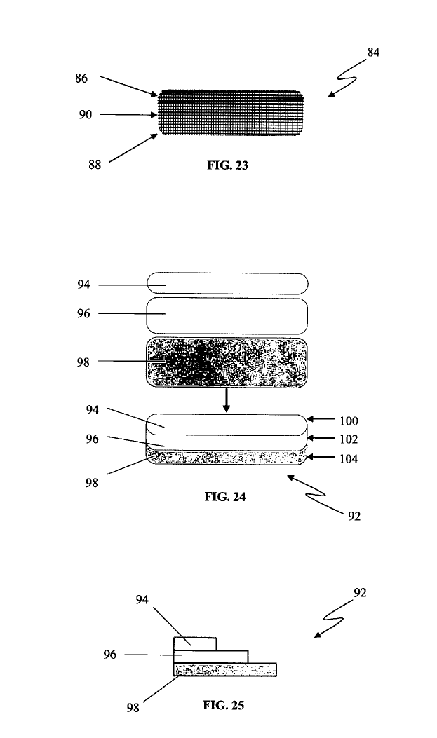

FIG. 24 illustrates still another example of an optional contoured single layer region 92 of the spacer 12 according to the present invention. In this example, fabric layers of different shapes are stacked consecutively on top of one another to form sections having different heights. As shown by way of example only, a first layer 94 is stacked on top of a second layer 96 and those two layers 94, 96 are then stacked on top of a third layer 98. Consequently, three sections having different heights are formed on single layer region 92. The first section 100 is formed from a first set of stacked fabric layers (layer 94 and the portions of layers 96, 98 disposed under layer 94), the second section 102 is formed from a second set of fabric layers (portions of layers 96, 98), and a third section 104 formed from a third set of fabric layers (a portion of layer 98). The key is that layer region 92 results in a textile layer having a contoured cross-sectional shape (e.g. tapered) due to the sections of varying height 100-104 that are formed from the different sets of fabric layers 94-98. FIG. 25 illustrates a side view of the end result of the layer region 92 having a generally tapered cross-sectional shape due to the varying quantities of fabric layers 94-98 that are stacked on top of one another.

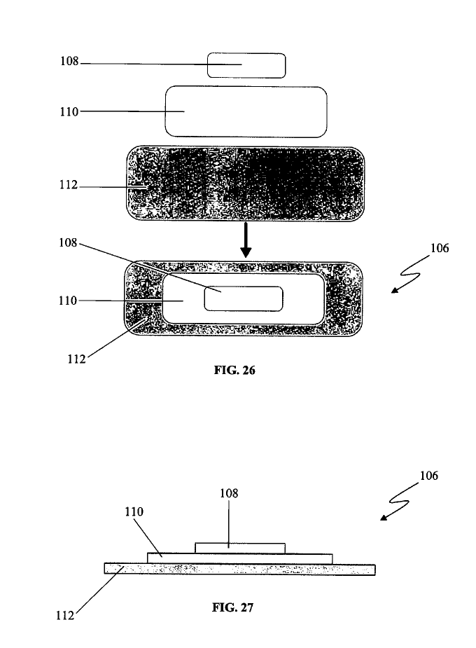

By way of example only, FIG. 26 illustrates another example of an optional contoured single layer region 106 showing fabric layers 108-112 having different sizes and different placements on the layer region 106 relative to the placement of layers 94-98 in FIGS. 24 & 25. As illustrated in the side cross-sectional view in FIG. 27, a dome shape can be achieved on layer region 106 by stacking the three fabric layers 108-112 of varying sizes on top of one another and centrally aligning the fabric layers 108-112 on layer region 106.

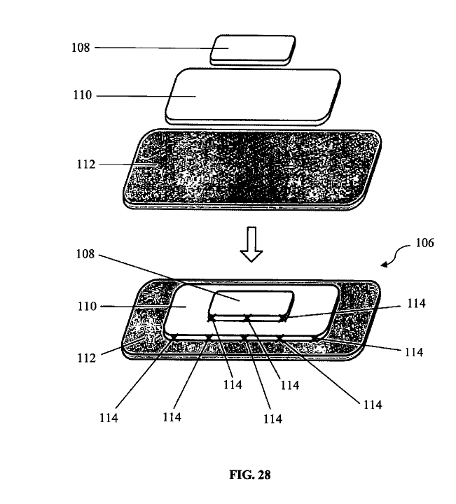

As shown for example in FIG. 28, after the three fabric layers 108-112 are stacked on top of one another, they may be coupled together via supplemental stitches 114 to hold the fabric layers 108-112 together and to maintain the contour of the dome shape on layer region 106. Although not shown, it is contemplated that the fabric layers 108-112 may be coupled together for example, via the use of hinge regions other fixation or coupling mechanisms, including but not limited to adhesives capable of coupling adjacent or multiple fabric layers together.

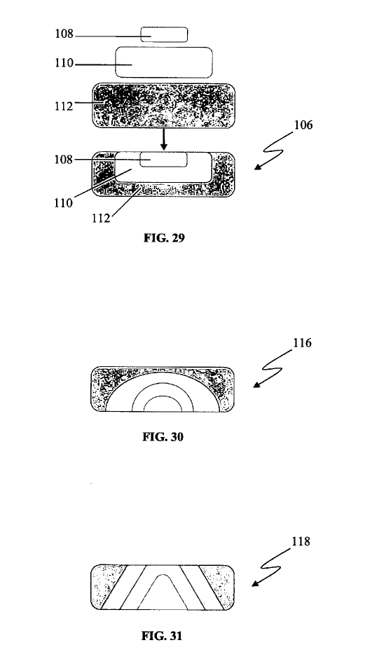

As shown for example in FIG. 29, the placement of fabric layers 108-112 on layer region 106 may also be varied in order to align the dome shape of layer region 106 according to the contour of any intervertebral disc space. By way of example only, as shown in FIGS. 30 & 31, more complex designs can be readily generated using various fabric layers to produce custom profiles on a single layer region. Specifically, FIG. 30 illustrates a single layer region 116 having a semi-circular design and FIG. 31 illustrates a single layer region 118 having a triangular design.

Although the different contours in FIGS. 24-31 are shown according to particular examples, it will be appreciated that these contours are not limited to these examples and can be applied to any other example and/or method described herein without departing from the scope of the present invention. In all instances, the contour of the various single layer regions described herein will collectively result in a spacer 12 having an anatomically similar shape to an intervertebral disc, thereby restoring the height of the intervertebral disc space while advantageously preserving the natural motion of the spine.

In some instances, it may be advantageous that each single layer region 22 of the spacer 12 be contoured to achieve an overall tapered shape, as shown in the cross-sectional view of the spacer 12 in FIG. 14. Thus, several examples and/or methods of achieving a generally tapered shape of the implant 10 and specifically the spacer 12 will now be described.

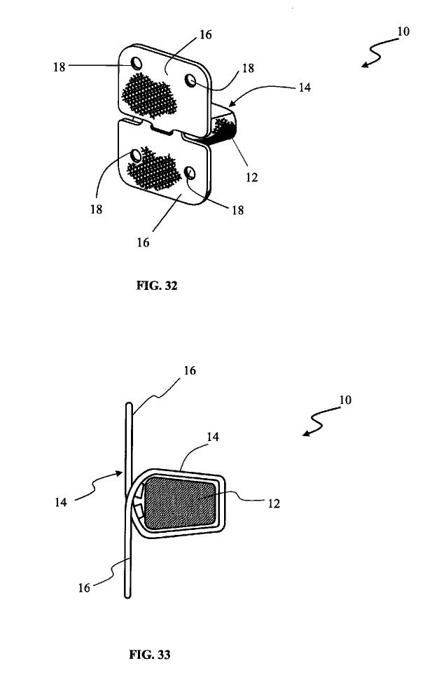

FIGS. 32-33 illustrate an alternative example of a tapered textile-based implant 10 according to one exemplary embodiment of the present invention. Implant 10 includes a tapered textile-based core 12 disposed within an encapsulating jacket 14. The encapsulating jacket 14 includes a pair of flanges 16 and a plurality of apertures 18 for receiving a fixation element (e.g. screw, staple, tack, suture, etc.) The tapered textile-based implant 10 of the present invention is suitable for use in a variety of surgical applications, including but not limited to spine surgery. When applied to spinal surgery and implanted into an intervertebral disc space, the tapered shape of the textile-based implant 10 advantageously forces the adjacent vertebral bodies into an angled relationship, thereby restoring (partially or fully) the natural curvature of the spine at that vertebral level. More specifically, the tapered cross-sectional shape of the implant 10 is designed to match the natural lordotic and/or kyphotic angles in any given region of the spine (i.e. lordosis in the cervical and lumbar regions of the spine and kyphosis in the thoracic region of the spine).

A variety of techniques may be used to form the tapered cross-sectional shape of the textile-based implant 10 of the present invention. Although described herein largely in terms of restoring lordosis in the lumbar or cervical spinal regions (i.e. with the implant height greater at the anterior portion than in the posterior portion), it will be understood that the taper may also be reversed to match the kyphotic curvature of the thoracic spine.

FIGS. 34-39 collectively illustrate an example of a first method for creating a tapered textile-based core 12 according to the present invention. In this method, threads of varying thickness or diameter are used to form regions of fabric having different height to form a tapered textile-based core 12 of the type shown in FIG. 33. The first step in the method involves manufacturing a base textile structure 120 as shown in FIG. 34. The base textile structure 120 is preferably manufactured via an embroidery process using any number of biocompatible filament materials (including but not limited to polyester thread). According to this embodiment, the biocompatible filament material includes a relatively large diameter thread 122, a relatively small diameter thread 124, and an intermediate diameter thread 126. Threads 122, 124, 126 are embroidered to form a plurality of layer regions, for example first layer region 128, second layer region 130, third layer region 132, and fourth layer region 134. The layer regions 128-134 are connected together in side-by-side relation via one or more of the threads 122, 124, 126 and separated by a distance to form a plurality of hinge regions 136a-136c between the layer regions 128-134, respectively.

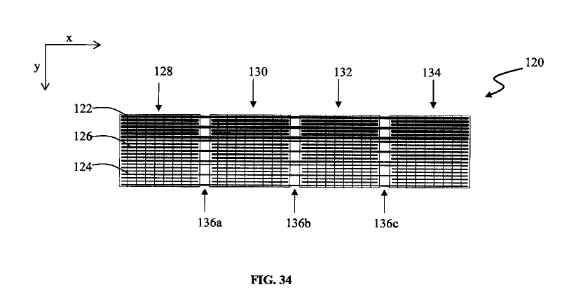

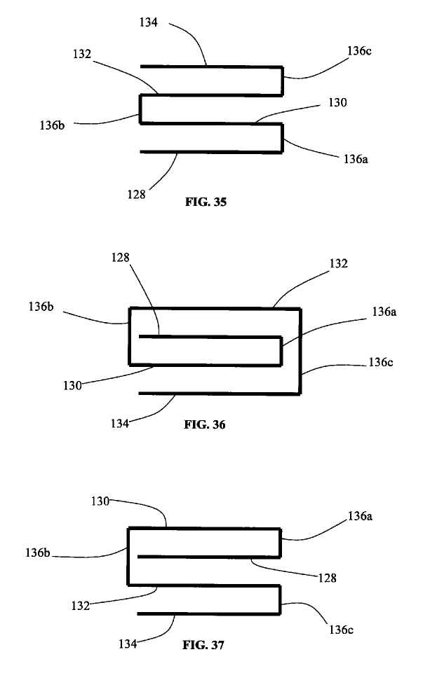

The next step in the first method of creating a tapered textile-based implant of the present invention involves folding the layer regions 128-134 of the base textile structure 120 at the hinge regions 136a-136c so that the layer regions 128-134 are stacked on top of each other. As will be described in detail below, the folding process may be performed in any number of manners, including back-and-forth folding (e.g. FIG. 35), one-way folding (e.g. FIG. 36), or a combination of both (e.g. FIG. 37). The key is that the layer regions 128-134, after being stacked on top of each other via any of these folding techniques, collectively result in a textile structure having a tapered cross-sectional shape due to the regions of varying thread diameter 122, 124, 126 that are stacked on top of one another.

FIGS. 35-37 illustrate examples of various folding techniques capable of producing the tapered textile-based implant 10 of the present invention. FIGS. 35-37 are cross-sectional views taken along the x-axis in FIG. 34 after folding four layer regions of the base textile structure of FIG. 34. FIG. 35 illustrates the core 12 after folding the layer regions 128-134 in a back-and-forth or accordion-like manner, with layer region 130 folded on top of layer region 128, layer region 132 folded on top of layer region 130, and layer region 134 folded on top of layer region 132. FIG. 36 illustrates the core 12 after folding the layer regions 128-134 in a one-way manner, with layer region 130 folded under layer region 128, layer region 132 folded over layer region 128, and layer region 134 folded under layer region 130. It will be appreciated that this may be reversed without departing from the scope of the invention, with layer region 130 folded over layer region 128, layer region 132 folded under layer region 128, and layer region 134 folded over layer region 130. FIG. 37 illustrates the core 12 after folding the layer regions 128-134 in a combination of back-and-forth and one-way manners, wherein (by way of example only) layers 128-132 are folded in a one-way manner (e.g. layer region 130 is folded over layer region 128 and layer region 132 is folded under layer region 128), while the layer region 134 is folded in a back-and-forth manner relative to layer region 132 (e.g. layer region 134 folded under layer region 132).

It will be appreciated that the lengths of the threads 122-126 forming the hinge regions 136a-136c may vary depending upon the folding technique employed. For example, the hinge regions 136a-136c of FIG. 35 may be generally the same length, while the hinge regions 136a-136c of FIGS. 36-37 may be different depending upon the distance between the respective layer regions 128-134 after folding. Varying the length of the hinge regions 136a-136c may also be advantageous in ensuring a compact construction after folding (i.e. causing the layer regions 128-134 to be in close proximity to one another after folding). The distances between the layer regions 128-134 in FIGS. 35-37 are exaggerated for the sake of clarity in explaining the folding techniques. It will be appreciated that the distances between the layer regions 128-134 in the textile-base core 12 will be smaller such that the textile-based core 12 will be far more compact in practice.

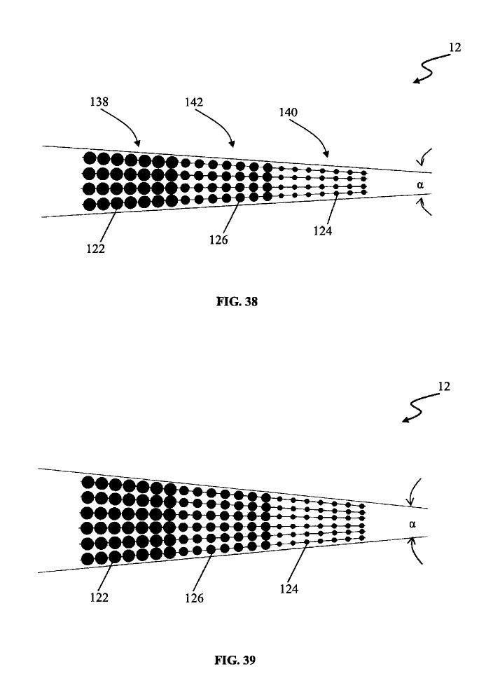

In all instances, it will be appreciated that folding layer regions 128-134 collectively result in a textile structure 12 having a tapered cross-sectional shape due to the regions of varying thread diameter 122, 124, 126 that are stacked on top of one another. This is best shown in FIG. 38, which is a cross-sectional view along the y-axis in FIG. 34 of the tapered textile-based core 12 after folding the four layer regions 128-134 of the base textile structure 120. By way of example only, the implant 12 is shown as folded via the back-and-forth method with layer regions 128-134 stacked consecutively on top of one another. Each tapered textile-based core 12 includes a first region 138 formed from the layers of large diameter thread 122, a second region 140 formed from the layers of small diameter thread 124, and a third region 142 formed from the layers of intermediate thread 126. By varying the respective and/or relative heights of the regions 138-142, the textile-based core 12 may be dimensioned with varying heights and/or angles to accommodate a wide variety of surgical requirements. The tapered textile-based core 12 may be provided in any number of suitable dimensions depending upon the surgical application and patient pathology. By way of example only, the angle (α) of the taper of the textile-based core 12 may be in the range from 1 to 122 degrees, and the heights of the regions 138-142 may range from 1 to 120 millimeters.

FIGS. 40-43 collectively illustrate an example of a second method for creating a tapered textile-based core 12 according to the present invention. In this method, a plurality of layer regions 144-150 of varying length are stacked upon one another to form a tapered textile-based core 12 of the type shown in FIG. 33. The first step in the method involves manufacturing a base textile structure 152 as shown in FIG. 40. The base textile structure 152 is preferably manufactured via an embroidery process using any number of biocompatible filament materials (including but not limited to polyester thread). According to this embodiment, the biocompatible filament material has the same diameter thread throughout. The thread is embroidered to form a plurality of layer regions with varying lengths, for example first layer region 144, second layer region 146, third layer region 148, and fourth layer region 150. The layer regions 144-150 are connected together in side-by-side relation and separated by a distance to form a plurality of hinge regions 154a-154c between the layer regions 144-150, respectively.

The next step in the second method of creating a tapered textile-based implant involves folding the layer regions 144-150 of the base textile structure 152 at the hinge regions 154a-154c such that the layer regions 144-150 are stacked on top of each other. As described in detail above, the folding process may be performed in any number of manners, including back-and-forth folding, one-way folding, or a combination of both. The key is that the layer regions 144-150, after being stacked on top of each other via any of these folding techniques, collectively result in a textile-based structure 12 having a tapered cross-sectional shape due to the varying height of the layer regions 144-150 after being stacked on top of one another.

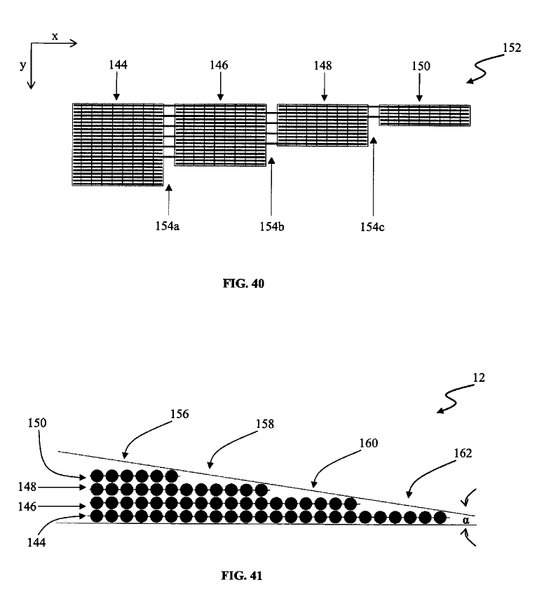

This is best shown in FIG. 41, which a cross-sectional view along the x-axis in FIG. 40 of the tapered textile-based core 12 after folding the four layer regions 144-150 of the base textile structure 152. By way of example only, the implant 12 is shown as folded via the back-and-forth method with layer regions 144-150 stacked consecutively on top of one another. Each tapered textile-based core 12 includes a first region 156 formed from a first set of predetermined fabric layers (e.g. four, including layer region 150 and the portions of layer regions 144-148 disposed under layer region 150), a second region 158 formed from a second set of predetermined fabric layers less than the first set (e.g. three, including portions of layer regions 144-148), a third region 160 formed from a third set of predetermined fabric layers less than the second set (e.g. two, including portions of layer regions 144-146), and a fourth region 162 formed from a fourth set of predetermined fabric layers less than the third set (e.g. one, including a portion of fourth region 146). By varying the respective and/or relative heights of the regions 156-162, the textile-based core 12 may be dimensioned with varying heights and/or angles (a) to accommodate a wide variety of surgical requirements.

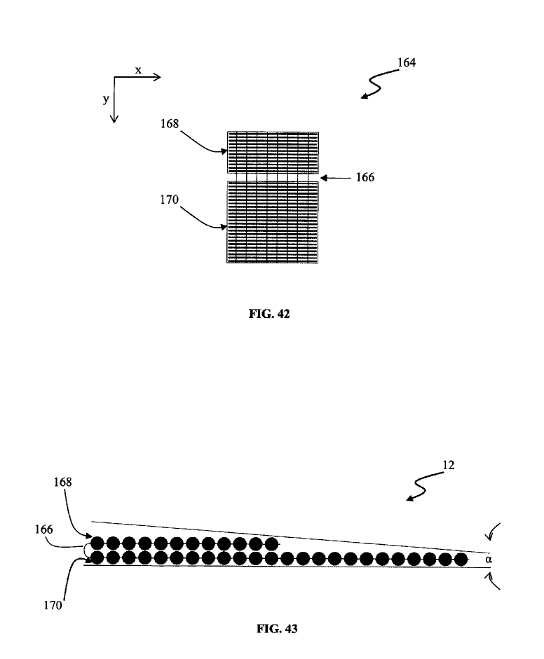

Regardless of the folding technique (back-and-forth, one-way, or a combination of both), the folding direction of each base textile structure 120 and 152 is generally along the x-axis of FIG. 34 and FIG. 40, respectively. It will be appreciated, however, that depending upon the base structure, the folding direction may be performed along the y-axis without departing from the scope of the present invention. For example, as shown in FIG. 42, a base textile structure 164 may be provided having two layer regions 168, 170 disposed generally co-linearly along the y-axis with a hinge region 166 therebetween. The layer regions 168, 170 may be folded with the layer region 168 on top of layer region 170 as shown in FIG. 43 (or vice versa). In all instances, it will be appreciated that the folding of layer regions 168, 170 will collectively result in the textile structure 12 having a tapered cross-sectional shape (with angle (α)) due to the varying quantities of layer regions 168, 170 that are stacked on top of one another.

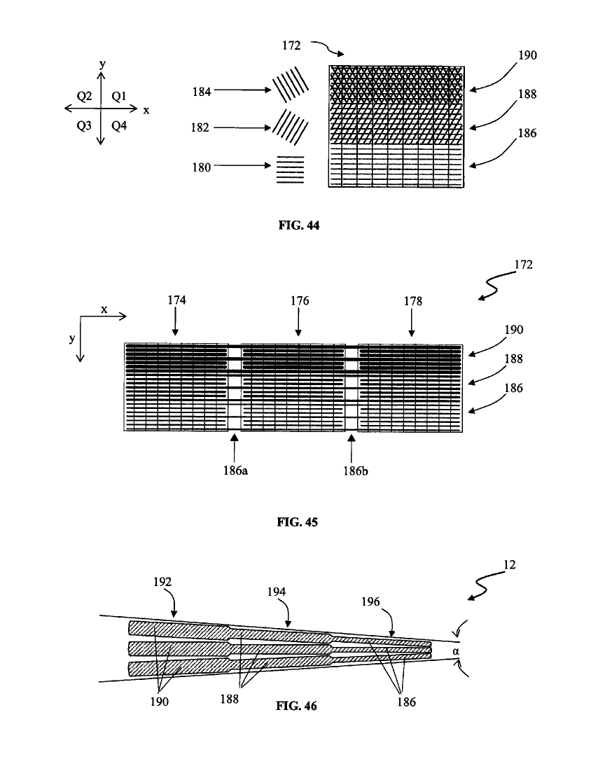

FIGS. 44-49 collectively illustrate an example of a third method for creating a tapered textile-based core 12 according to the present invention. In this method, sections of varying stitch density are used to form regions of fabric having varying height to form a tapered textile-based core 12 of the type shown in FIG. 33. The first step in the method involves manufacturing a base textile structure 172 as shown in FIG. 44. The base textile structure 172 is preferably manufactured via an embroidery process using any number of biocompatible filament materials (including but not limited to polyester thread). According to this embodiment, as shown in FIG. 45, each layer region 174, 176, 178 of the base textile structure 172 is constructed from biocompatible filament material including a first layer of stitching 180 having individual stitches oriented along the x-axis, a second layer of stitching 182 having individual stitches oriented angularly within the first and third quadrants (Q1, Q3) of the x-y plane, and a third layer of stitching 184 having individual stitches oriented angularly within the second and fourth quadrants (Q2, Q4) of the x-y plane. The individual layer regions 174-178 of the base textile structure 172 are coupled together via hinge regions 186a-186b formed by one or more threads laid down during the embroidery process.

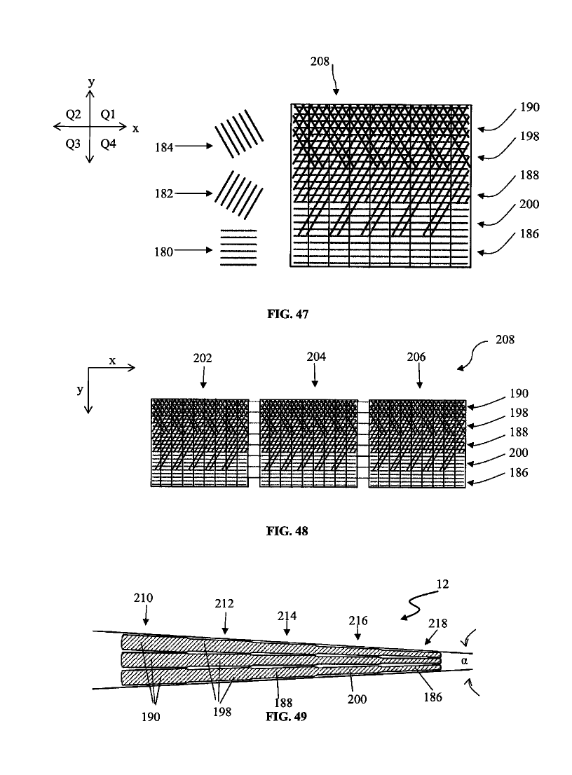

The lengths of the stitching layers 180-184 (i.e. distance along the y-axis) may vary depending upon the desired taper angle (α). For example, the stitching layer 180 extends the entire length of the layer region 174-178, the stitching layer 182 extends a shorter distance than the stitching layer 180 (e.g. approximately ⅔^{rd} the length of the layer region 174-178), and the stitching layer 184 extends a shorter distance than the stitching layer 182 (e.g. approximately ⅓^{rd} the length of the layer region 174-178). As will be appreciated, the length of the stitching layers 180-184 may be varied from that shown in FIGS. 44-45 without departing from the scope of the invention, such as (by way of example only) shown in FIGS. 47-48 described below.

Arranging the stitching layers 180-184 in this manner advantageously forms sections of varying density, for example first section 186 comprising the first layer of stitching 180, second section 188 comprising the second layer 182 stitched on top of the first layer 180 to form double stitching, and third section 190 comprising the third layer 184 stitched on top of the first and second layers 180 and 182 to form triple stitching. In addition to providing the ability to stack the layer regions 174-178 to create a tapered textile-based implant 12 (as will be described below), varying the stitch density in this manner also advantageously minimizes stitch consolidation, increases porosity, and enhances elasticity of the implant 12.

The next step in the third method of creating a tapered textile-based implant 12 of the present invention involves folding the layer regions 174-178 of the base textile structure 172 at the hinge regions 186a-186b such that the layer regions 174-178 are stacked on top of each other. As described in detail above, the folding process may be performed in any number of manners, including back-and-forth folding, one-way folding, or a combination of both. The key is that the layer regions 174-178, after being stacked on top of each other via any of these folding techniques, collectively result in a textile structure 12 having a tapered cross-sectional shape due to the sections of varying density 186, 188, 190 that are stacked on top of one another.

This is best shown in FIG. 46, which is a cross-sectional view along the y-axis in FIG. 44 of the tapered textile-based core 12 after folding the three layer regions 174-178 of the base textile structure 172. By way of example only, the implant 12 is shown as folded via the back-and-forth method with layer regions 174-178 stacked consecutively on top of one another. Each tapered textile-based core 12 includes a first region 192 formed from the layers of triple stitching 190, a second region 194 formed from the layers of double stitching 188, and a third region 196 formed form the layers of single stitching 186. By varying the respective and/or relative heights of the regions 192-196, the textile-based core 12 may be dimensioned with varying heights and/or angles to accommodate a wide variety of surgical requirements.

By way of example only, FIG. 47 illustrates a base textile structure 172 having five (5) sections of varying density, the three sections 186, 188, 190 from the embodiment shown in FIGS. 44-46 along with two new sections 198, 200. The additional sections of varying density are created by alternating the stitch paths. For example, as shown in FIG. 48, alternate double stitch paths are omitted in sections 198 and 200 to create two more different densities than in the embodiment shown in FIGS. 44-46. As a result, five sections of different densities 190, 198, 188, 200, 186 are formed out of the same three stitching orientations 180, 182, 184. As best shown in FIG. 49, increasing the number of sections of varying density reduces the height differential between the adjacent sections of varying density 190, 198, 188, 200, 186. This, in turn, causes the textile-based core 12 to have a tapered shape with a smoother gradient profile than shown in FIG. 46.

This is best shown in FIG. 49, which is a cross-sectional view along the y-axis in FIG. 48 of the tapered textile-based core 12 after folding the three layer regions 202, 204, 206 of the base textile structure 208. By way of example only, the implant 12 is shown as folded via the back-and-forth method with layer regions 202-206 stacked consecutively on top of one another. Each tapered textile-based core 12 includes a first region 210 formed from the layers of triple stitching 190, a second region 212 formed from the additional layers 198, a third region 214 formed from the layers of double stitching 188, a fourth region 216 formed from the additional layers 200, and a fifth region 218 formed form the layers of single stitching 186. By varying the respective and/or relative heights of the regions 210-218, the textile-based core 12 may be dimensioned with varying heights and/or angles to accommodate a wide variety of surgical requirements.

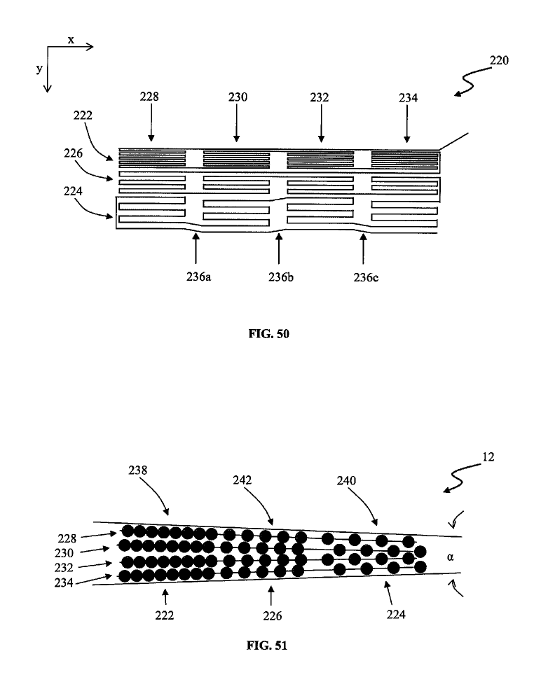

FIGS. 50-51 collectively illustrate an example of a fourth method for creating a tapered textile-based core 12 according to the present invention. In this method, sections of different yarn spacing are used to form regions of fabric having different height to form a tapered textile-based core 12 of the type shown in FIG. 33. The first step in the method involves manufacturing a base textile structure 220 as shown in FIG. 50. The base textile structure 220 is preferably manufactured via an embroidery process using any number of biocompatible filament materials (including but not limited to polyester thread). According to this embodiment, the biocompatible filament material has the same diameter thread throughout. The thread is embroidered to form sections of different yarn spacing, for example a tightly-spaced section 222, a loosely-spaced section 224, and an intermediately-spaced section 226. Sections 222-226 are embroidered to form a plurality of layer regions, for example first layer region 228, second layer region 230, third layer region 232, and fourth layer region 234. The layer regions 228-234 are connected together side-by-side relation and separated by a distance to form a plurality of hinge regions 236a-236c between the layer regions 228-234, respectively.

The next step in the third method of creating a tapered textile-based implant of the present invention involves folding the layer regions 228-234 of the base textile structure 220 at the hinge regions 236a-236c such that the layer regions 228-234 are stacked on top of each other. As described in detail above, the folding process may be performed in any number of manners, including back-and-forth folding, one-way folding, or a combination of both. The key is that the layer regions 228-232, after being stacked on top of each other via any of these folding techniques, collectively result in a textile structure 12 having a tapered cross-sectional shape due to the sections of varying yarn spacing 222-226 that are stacked on top of one another.

This is best shown in FIG. 51, which a cross-sectional view along the y-axis in FIG. 50 of the tapered textile-based core 12 after folding the four layer regions 228-232 of the base textile structure 220. By way of example only, the implant 12 is shown as folded via the back-and-forth method with layer regions 228-234 stacked consecutively on top of one another. Each tapered textile-based core 12 includes a first region 238 formed from the layers of tightly-spaced yarn 222, a second region 240 formed from the layers of loosely-spaced yarn 224, and a third region 242 formed from the layers of intermediately-spaced yarn 226. By varying the respective and/or relative heights of the regions 238-242, the textile-based core 12 may be dimensioned with varying heights and/or angles to accommodate a wide variety of surgical requirements.

FIGS. 52-54 collectively illustrate an example of a fifth method for creating a tapered textile-based core 12 according to the present invention. In this method, fabric layers having different heights (along x-axis) and widths (along y-axis) are used to form a tapered textile-based core 12 of the type shown in FIG. 33. The first step in the method involves manufacturing a base textile structure 244 as shown in FIG. 52. The base textile structure 244 is preferably manufactured via an embroidery process using any number of biocompatible filament materials (including but not limited to polyester thread). According to this embodiment, the biocompatible filament material has the same diameter thread throughout. The thread is embroidered to form a plurality of layer regions 246, 248, 250, 252, 254, 256, 258, 260, 262, 264, 266, 268, 270, 272 with gradually increasing heights and widths. The layer regions 246-272 are connected in side-by-side relation and separated by a distance to form a plurality of hinge regions 274a-274m between the layer regions 246-272, respectively.

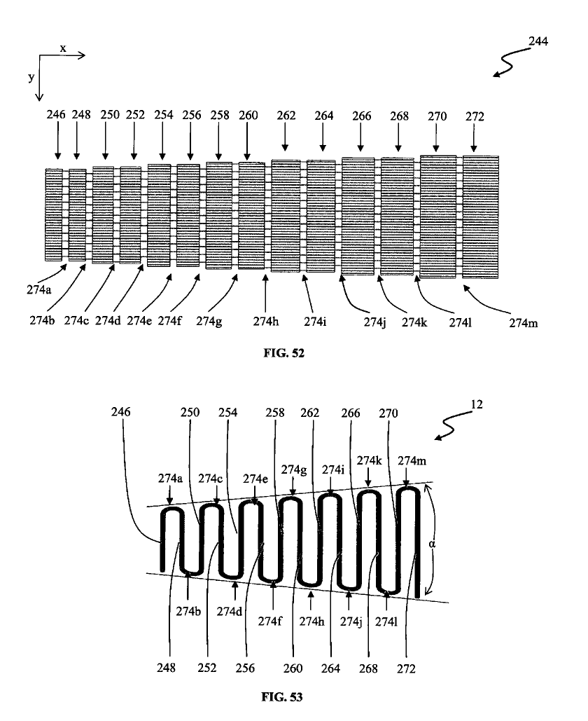

The next step in the fifth method of creating a tapered textile-based implant of the present invention involves folding the layer regions 246-272 of the base textile structure 244 at the hinge regions 274a-274m in a back-and-forth manner so that the layer regions 246-272 are stacked together with the layer regions 246-272 oriented generally perpendicularly from their orientation of FIG. 52. As best shown in FIGS. 53-54, this forms a taper due to the increasing widths of the layer regions 246-272. The layer regions 246-272 are shown in a generally loosely spaced configuration to facilitate the explanation. However, it will be understood that the spacing of the layer regions 246-272 would be much closer in practice to ensure the implant 12 has sufficient strength to withstand the loading (e.g. axial) after implantation without undue deformation. The key is that the layer regions 246-272, after being stacked together, result in a vertically situated textile structure having a tapered cross-sectional shape due to the increasing height of the layer regions 246-272.

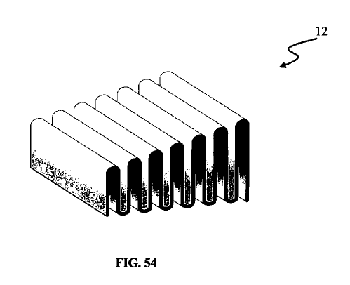

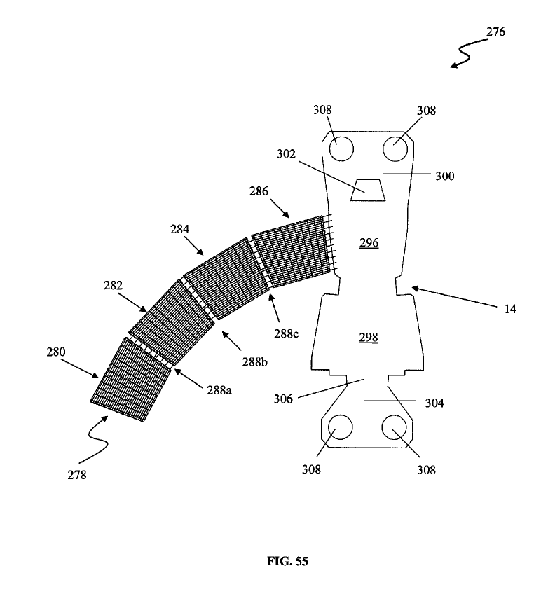

FIG. 55 illustrates an example of a sixth method for creating a tapered textile-based core 12 according to the present invention. In this method, a base textile structure 276 is manufactured having an unfolded base textile structure 278 attached to an unfolded encapsulating jacket 14. The base textile structure 276 is preferably manufactured via an embroidery process using any number of biocompatible filament materials (including but not limited to polyester thread). According to this embodiment, the biocompatible filament material has the same diameter thread throughout. It will be appreciated that the base textile structure 278 and encapsulating jacket 14 may be formed different materials and/or made from techniques other than embroidery without departing from the scope of the invention.

The base textile structure 278 may include any number of layer regions without departing from the scope of the present invention. For example, the base textile structure 278 may have four layer regions 280, 282, 284, 286 separated by hinge regions 288a-288c, respectively. Layer regions 280-286 may be manufactured using any number of different stitching or sewing paths. By way of example only, FIG. 56 illustrates an exemplary sewing path for creating the base textile structure 278. The layer regions 280-286 are shown in a generally straight line along the x-axis, it will be appreciated that the layer sections 280-286 may be formed in a generally arcuate manner as shown in FIG. 55. The sewing path of the core thread 290 begins at point 292 and ends at point 294. In order to facilitate ease of manufacture, lines of extra flexibility are introduced into the embroidered assembly. The base textile structure 278 may then be readily folded and placed within a jacket with accuracy and reproducibility. In this example, the thick core thread 290 joins the adjacent four layer regions 280-286, one thread in three, creating flexible hinge regions 288a-288c, between the layer regions 280-286, respectively. The hinge regions 288a-288c facilitate folding of the layer regions 280-286.

As described in detail above, the folding process may be performed in any number of manners, including back-and-forth folding, one-way folding, or a combination of both. The key is that the layer regions 222-226, after being stacked on top of each other via any of these folding techniques, collectively result in a textile structure 12 having a tapered cross-sectional shape due to the sections 280-286 that are stacked on top of one another.

The jacket 14 includes a first section 296 and a second section 298. First section 296 includes a flange region 300 having an aperture 302 formed therein. Second section 298 includes a flange region 304 having a generally triangular shape with a narrow region 306. After the layer regions 280-286 are stacked, the resulting core 12 is folded onto the first section 296. At that point, the second section 298 is folded over the top of the stacked layer regions 280-286 and the flange region 304 is passed through the aperture 302 of the flange region 300. According to one embodiment, one or more of the edges of the first section 296 and second section 298 may be stitched together to encapsulate the stacked layer regions 280-286 therein. The flange regions 300, 304 extend from the encapsulated jacket 14 such that they can be used for affixing the encapsulated jacket 14 to adjacent anatomic structures (e.g. adjacent vertebral bodies) to maintain the implant 10 in position before the embroidery becomes encapsulated with scar tissue. Apertures 308 may be optionally provided in the flanges 300, 304 to accommodate anchors, such as a screw or other means of fixation, which secure the textile-based implant 10 to the adjacent spinal vertebrae.

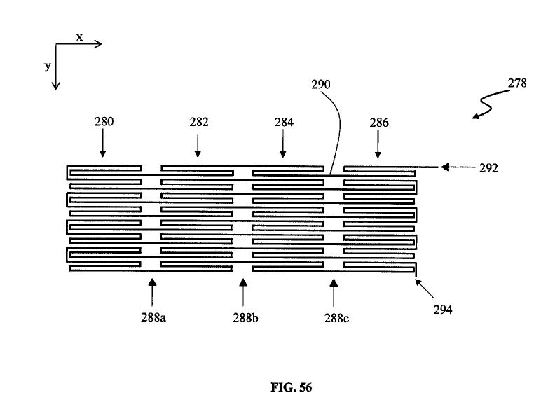

FIGS. 57-60 illustrate examples of the spacer 12 containing an internal radio-opaque marker or markers, according to one embodiment of the present invention. By way of example only, FIG. 57 includes a single radio-opaque marker 310 in the form of an elongated cylinder, and FIG. 58 includes three radio-opaque markers 312 in the form of smaller cylinders. The radio-opaque markers 310, 312 may be composed of any kind of radio-opaque material including but not limited to metal (e.g. titanium, stainless steel, etc.). Radio-opaque markers 310, 312 are advantageous when tracking the implant 10 post-surgery and intra-operatively via fluoroscopy. Although shown as having a generally cylindrical shape, the radio-opaque marker 310, 312 may take many shapes including but not limited to generally square, rectangular, spherical, oval, ring, and polygonal. It will be appreciated that the number of radio-opaque markers (i.e. one marker 310 in FIG. 57 and three markers 312 in FIG. 58) is set forth by way of example only and that the number may be increased or decreased without departing from the scope of the present invention. Accordingly, layer regions 22 of the spacer 12 may contain slots 314 or holes 316 to accommodate any number, shape or size of radio-opaque markers 310, 312, as illustrated in FIGS. 59-60. The radio-opaque markers 310, 312 may then be stitched onto the layer regions 22.

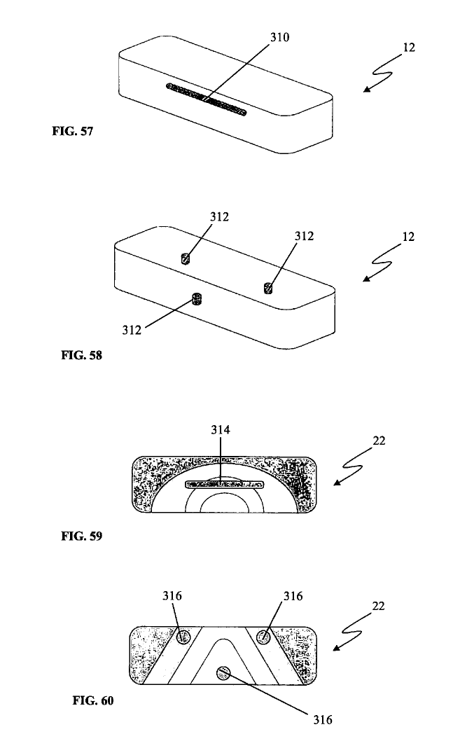

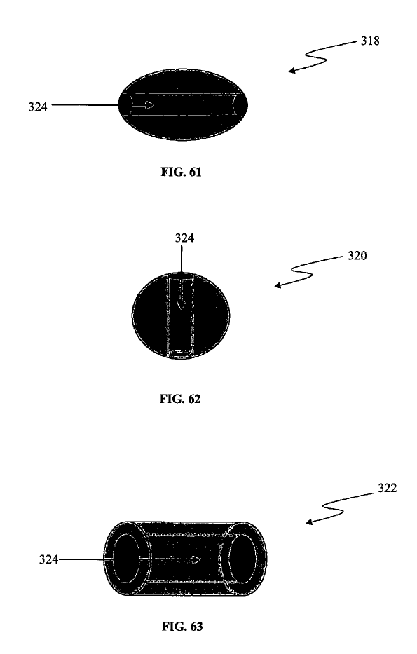

As shown in FIGS. 61-63, the radio-opaque marker may also take the form of a bead 318, 320, 322 that includes a hole 324 through its center. In this way, a stitching thread can pass through the center hole 324 of the radio-opaque bead 318, 320, 322 and secure it into position within the textile spacer. It will be appreciated that the elliptical, spherical, and tube shapes of the radio-opaque beads 318, 320, 322 shown in FIGS. 61, 62, and 63, respectively, are set forth by way of example only and that the shape of the radio opaque bead may change without departing from the scope of the present invention.

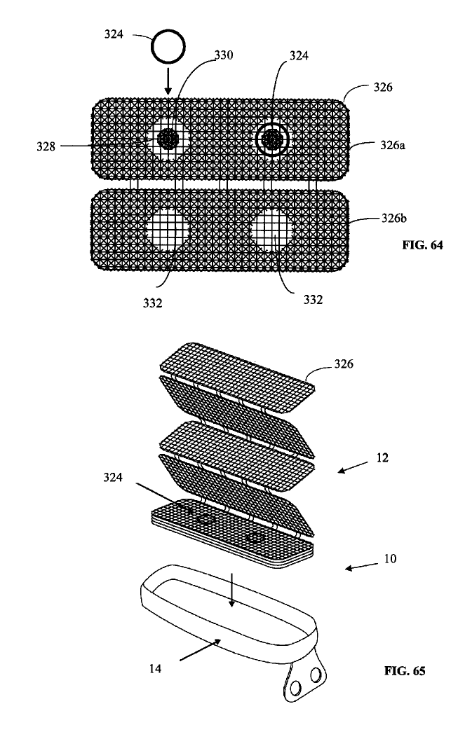

FIGS. 64-74 illustrate another example of a spacer 12 including a radio-opaque marker 324 according to the present invention. By way of example only, radio-opaque marker 324 comprises a ring marker positioned within the spacer 12. The radio-opaque marker 324 may be composed of any kind of radio-opaque material including but not limited to metal (e.g. titanium, stainless steel, etc.). FIG. 64 illustrates a portion of an example of a base textile structure 326 forming part of the spacer 12 according to one embodiment of the present invention. In the example shown in FIG. 64, the spacer 12 is provided with a pair of marker rings 324 positioned collinearly in each half of the spacer 12. However, any number and configurations of the marker ring 324 may be used. Two (or more) adjacent single layer regions 326a, 326b are specifically contoured to provide the housing for the marker ring 324. As will be described in further detail below, the first single layer region 326a includes a recess 328 having an embroidered peg 330 located centrally therein. The second adjacent single layer region 326b includes a recess 332 positioned opposite the recess 328 so as to form a generally cylindrical pocket with recess 328 for snugly housing the marker ring 324 when folded as shown in FIG. 65 (illustrating the base textile structure 326 having marker rings 324 being folded into spacer 12 and inserted into jacket 14 to form implant 10).

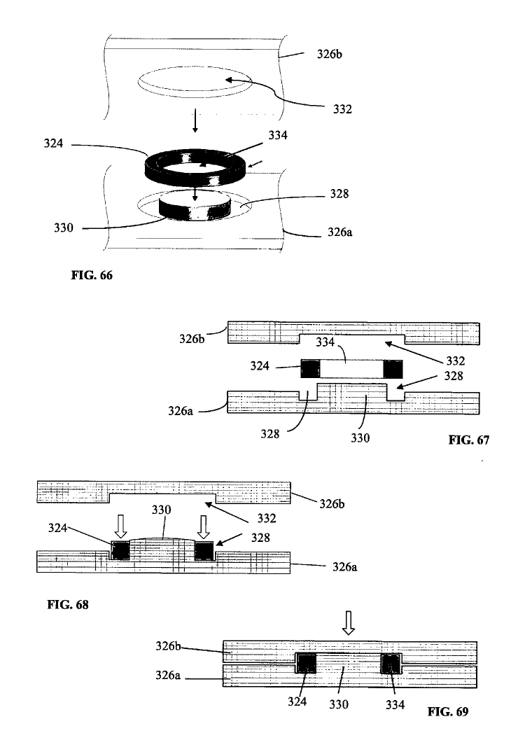

FIGS. 66-69 illustrate in further detail the example of the placement of the marker ring 324 between layer regions 326a, 326b. Layer region 326a is manufactured via an embroidery process in a manner that allows the formation of the peg 330 within a recess 328. This process is discussed in greater detail below. The peg 330 extends generally perpendicularly from the recess 328 and extends farther than the surface of the layer region 326a (such that it “sticks out” from the recess 328. The marker ring 324 includes a central aperture 334 that has a diameter (or size) that is slightly smaller than the diameter (or size) of the peg 330, as best shown in FIG. 67. This creates an interference fit between the marker ring 324 and peg 330 that is possible largely due to the compressibility of the textile material making up the peg 330. This push interference fit helps secure the marker ring 324 in place. Additional sutures (not shown) may also be sewn around the marker ring 324 to secure it within the recess 328. The peg 330 is further dimensioned to extend partially beyond the edge of the marker ring 324 when the marker ring 324 is fully inserted onto the peg 330, as best shown in FIG. 68. This enables the peg 330 to tightly engage the second layer region 326b within recess 332. The recess 328 is dimensioned such that the marker ring 324 fits relatively snugly therein. The recess 332 on the second layer region 326b is dimensioned to fit relatively snugly around the marker ring 324 once the layer regions 326a, 326b have been folded on top of one another, as shown in FIG. 69.

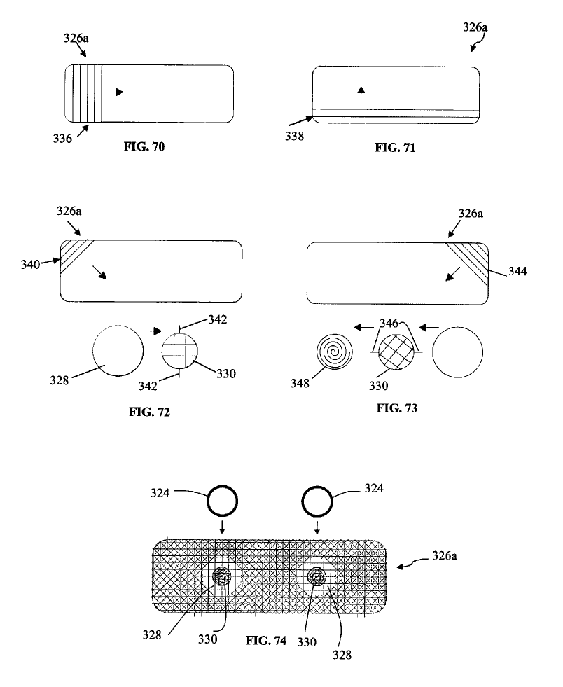

FIGS. 70-74 provide one example of an embroidery process used in forming single layer region 326a containing recess 328 and peg 330. The process described herein provides for accurate and repeatable position of the marker ring(s) 324. As a first step, shown in FIG. 70, transverse stitches 336 having low-extension 8 mm stitch length paths are placed on the single layer region 326a proceeding in a left-to-right (in FIG. 70) direction, including the area that will become the recess 328. As a second step, shown in FIG. 71, longitudinal stitches 338 having low-extension 8 mm stitch length paths are placed on the single layer region 326a proceeding in a bottom-to-top direction (in FIG. 71), including the area that will become the recess 328. The transverse and longitudinal stitches 336, 338 sewn in the first and second steps effectively create a backing layer, wherein the relatively long stitch lengths (8 mm by way of example only) ensure a dimensionally stable base layer.

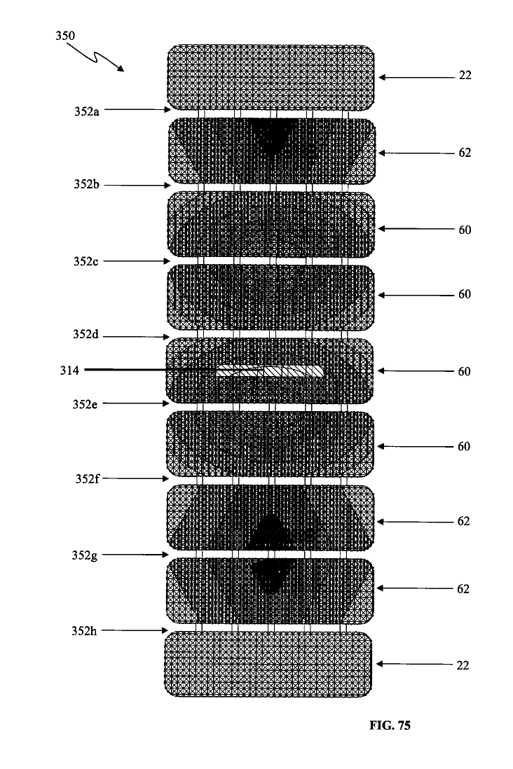

As a third step, shown in FIG. 72, diagonal stitches 340 having a 1.5 mm stitch length path are sewn on the single layer region 326a proceeding from the top-left to the bottom-right (in FIG. 72). The area that is to become recess 328 is left unstitched in this step. Also, stitching of the peg 330 is begun, with the horizontal and vertical elements stitched thereon. The threads 342 proved a bridge to the peg 330 from the top and bottom as shown. As a fourth step, shown in FIG. 73, diagonal stitches 344 having a 1.5 mm stitch length path are sewn on the single layer region 326a proceeding from the top-right to the bottom-left (in FIG. 72). The area that is to become recess 328 is left unstitched in this step. The relatively short stitch lengths on the diagonal stitches 340, 344 effectively hold the construct together. Also, stitching of the peg 330 continues with the diagonal elements stitched thereon. Threads 346 provide a bridge to the peg 330 from the sides as shown. The peg 330 may be optionally finished by adding spiral stitching 348 to give extra height and stability for holding the marker ring 324. The spiral stitching 348 may also facilitate engagement of the marker 324 and peg 330 by adding “give” to the peg 330 to aid in the interference fit described above. FIG. 74 illustrates a fully stitched single layer region 326a according the process described above, with marker rings 324 ready for placement within recesses 328.

In the example described above and shown in FIGS. 64-74, the marker ring 324 is shown as an annular ring disposed within annular recess 328, 332 and upon a generally cylindrical embroidered peg 330. However, it should be understood that the described shapes are provided by way of example only, and the radio-opaque marker 324 and corresponding structure within the spacer 12 may have any geometric shape without departing from the scope of the present invention. Moreover, the example shown and described above included a pair of marker rings 324 and a pair of corresponding structures within the spacer 12 (e.g. recess 328, peg 330, recess 332). While having a pair of radio-opaque markers 324 provides enhanced positional information during x-ray imaging (relative to only one marker), any number of markers 324 (and corresponding structures) may be used without departing from the scope of the present invention.

It will be appreciated that the spacer 12 may incorporate one or more or all of the features described herein and any combination thereof without departing from the scope of the present invention. In fact, layering many individual layer regions 22, each with different contours, achieves a complex structure that can more accurately conform to an intervertebral disc space. Combining individual layer regions 22 of different contours can create a spacer 12 having the precise anatomical features of an offset dome, a lordosis angle and a taper off at the side. With this layering method, an implant can replicate or approximate the exact anatomical shape and structure of an intervertebral disc. This method can then be applied to custom fit an implant to an individual patient in order to better treat his/her pathology. In all cases, it will be understood that the implant 10 restores the normal height of the intervertebral disc space, while advantageously preserving motion of the spine.

FIG. 75 illustrates an example of a base textile structure 350 having hinge regions 352a-352h and multiple single layer regions of different contours described above. By way of example only, base textile structure 350 includes a pair of non-contoured single regions 22 (e.g. FIG. 12), four semi-circular contoured single layer regions 60 (e.g. FIG. 20), one single layer region 60 including a slot 314 (e.g. FIG. 59) to accommodate an elongated radio-opaque marker (not shown), and three triangular contoured single layer regions 62 (e.g. FIG. 21). Once the radio-opaque marker is securely contained in the slot 314 via embroidery (or any other suitable means), the base textile structure 350 is then folded at hinge regions 352a-352h such that the layer regions 22, 60, 62 are stacked on top of one another to form the spacer 12. Alternatively, part of the base textile structure 350 may be folded first to form part of the spacer 12 and the radio-opaque marker may be securely contained onto that part of the spacer 12. Then, the rest of the base textile structure 350 may be folded on top of the radio-opaque marker to complete the spacer 12. With each layer region 22, 60, 62 having an orientation in relation to the final shape of the implant 10, the hinged design of the base textile structure 350 ensures that the layer regions 22, 60, 62 are correctly aligned when stacked on top of one another. By having the layer regions 22, 60, 62 hinged together, the correct combination, order, and orientation of the components will occur every time. This enables consistency and reproducibility in the manufacturing process of the spacer 12.

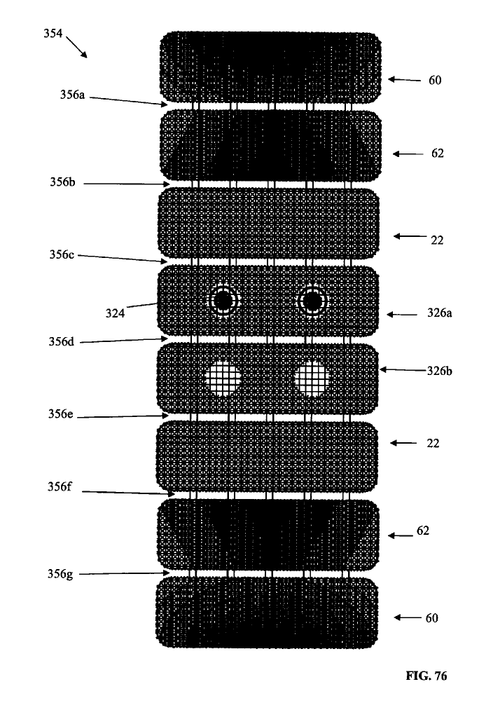

FIG. 76 illustrates an example of a base textile structure 354 having hinge regions 356a-356g and multiple single layer regions of different contours described above. By way of example only, base textile structure 354 includes a pair of non-contoured single regions 22 (e.g. FIG. 12), and a pair of triangular contoured single layer regions 62 (e.g. FIG. 21). The base textile structure 354 further includes single layer regions 326a, 326b for accommodating a radio-opaque ring marker 324 as described in relation to FIGS. 64-74 above. Once the radio-opaque marker 324 is securely contained, the base textile structure 354 is then folded at hinge regions 356a-356g such that the layer regions 22, 60, 62, 326a, 326b are stacked on top of one another to form the spacer 12. Alternatively, part of the base textile structure 354 may be folded first to form part of the spacer 12 and the radio-opaque marker may be securely contained onto that part of the spacer 12. Then, the rest of the base textile structure 354 may be folded on top of the radio-opaque marker to complete the spacer 12. With each layer region 22, 60, 62, 326a, 326b having an orientation in relation to the final shape of the implant 10, the hinged design of the base textile structure 350 ensures that the layer regions 22, 60, 62, 326a, 326b are correctly aligned when stacked on top of one another. By having the layer regions 22, 60, 62, 326a, 326b hinged together, the correct combination, order, and orientation of the components will occur every time. This enables consistency and reproducibility in the manufacturing process of the spacer 12

FIG. 77 illustrates an example of a base textile structure 358 having hinge regions 360, 362 and contoured layer regions 364, 366, 368. The layer regions 364-368 are similar to the single layer region 106 of FIGS. 26-28. Each layer region 364-368 has three different fabric layers 370, 372, 374 aligned to build a dome shape on the individual layer regions 364-368. When the base textile structure 358 is folded at hinge regions 360-362 such that the contoured layer regions 364-368 are stacked on top of one another to form a spacer 12, the resulting spacer 12 has an overall contoured dome shape. This dome shaped spacer 12 is anatomically similar in shape to an intervertebral disc. Therefore, once implanted, the dome shaped spacer 12 helps to restore the height of the intervertebral disc space and preserve the natural motion of the spine. As described above, the hinged design of the base textile structure 358 ensures that the layer regions 364-368 are correctly aligned when stacked on top of one another. In this manner, the hinged design enables consistency and reproducibility in the manufacturing process of the spacer 12.

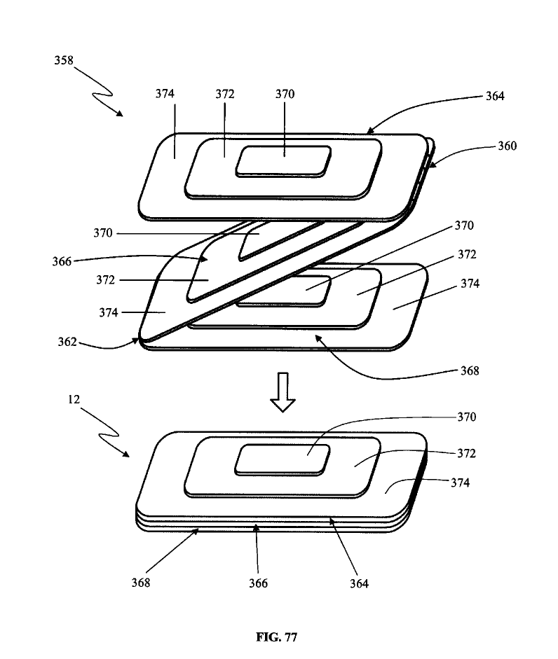

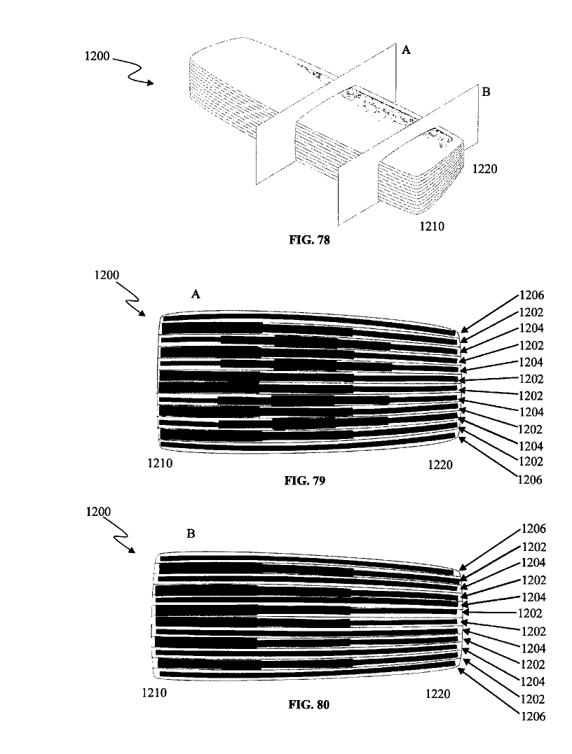

FIGS. 78-80 illustrate, by way of example, a spacer 1200 containing individually contoured layer regions 1202-1206. As a result of the individually contoured layer regions 1202-1206, the spacer 1200 has a contoured cross-sectional shape. More specifically, tapered layer regions 1202 and dome shaped layer regions 1204 are used to build the height of the spacer 1200. Flat layer regions 1206 are used as the top and bottom layers in order to impart smooth surfaces for the top and bottom of the spacer 1200. When tapered layer regions 1202 and dome shaped layer regions 1204 are stacked on top of one another, the precise anatomical features of an offset dome, an angle of curvature, and a taper off at the periphery are achieved. This is illustrated through the two different cross-sections in FIGS. 79 & 80.

For example, at a point in the middle of the spacer 1200, such as point A in FIG. 78, more height is needed in order to conform to the anatomical dimensions of an intervertebral disc space. As shown in the cross-sectional view of the spacer 1200 at point A in FIG. 79, more height is achieved in the middle of the spacer 1200 due to the convexity of the dome shaped layer regions 1204. At a point toward the end of the spacer 1200, such as point B in FIG. 78, less height is needed in order to conform to the anatomical dimensions of an intervertebral disc space. Accordingly, as shown in the cross-sectional view of the spacer 1200 at point B in FIG. 80, the convexity of the dome shaped layer regions 1204 diminishes at the periphery, causing the overall height of the spacer 1200 to be less at point B of the spacer (e.g. toward the ends of the spacer) than at point A (e.g. in the middle of the spacer).

As shown in both cross-sectional views A, B in FIGS. 79 & 80, a slight taper angle is formed throughout the spacer 1200 due to the individually tapered layer regions 1202. More specifically, the height of the spacer 1200 gradually decreases from one side 1210 of the spacer to the other side 1220 because each individual layer region 1202 gradually decreases from one side 1210 to the other side 1220. In this manner, the spacer 1200 is capable of matching the natural lordotic and/or kyphotic angles in any given region of the spine (e.g. lordosis in the cervical and lumbar regions of the spine and kyphosis in the thoracic region of the spine.) It will be appreciated that the arrangement of the contoured layer regions 1202-1206 (i.e. plain layer regions 1206 on the top and bottom surfaces and alternating tapered layer regions 1202 with dome shaped layer regions 1204) is set forth by way of example only in FIGS. 79 & 80, and may be varied without departing from the scope of the present invention.

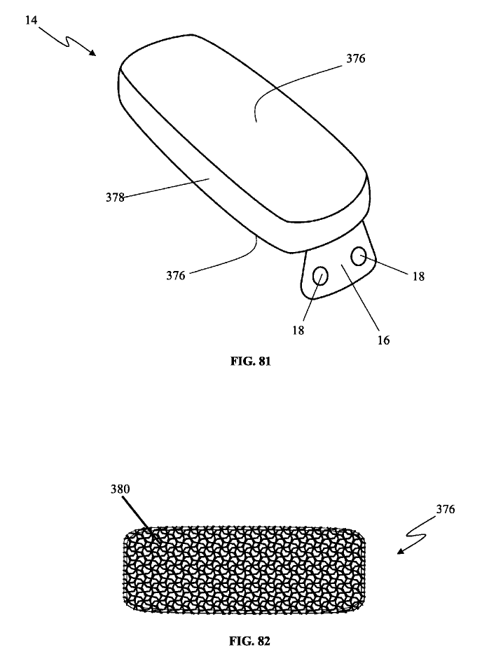

FIG. 81 illustrates an example of an encapsulating jacket 14 according to one embodiment of the present invention. In this preferred embodiment, the encapsulating jacket 14 comprises two outer caps 376 (i.e., one outer cap for the top of the implant and one outer cap for the bottom of the implant) and a circumferential barrier 378 with attachment flange 16. The outer caps 376 of the encapsulating jacket 14 may be designed to allow for expansion in order to accommodate any and all shapes of the spacer 12 described herein. By way of example only, the outer cap 376 may have a yarn path that is in a zigzag pattern 380 as shown in FIG. 82, and similar to that shown and described in commonly owned and co-pending PCT Application Serial No. PCT/US2008/052524 referenced above. The zigzag pattern 380 creates three dimensional outer caps 376 to cover the top and bottom of the complex anatomically matching spacer 12.

The outer caps 376 with a zigzag pattern 380 advantageously smooth out the surface of the textile spacer 12 such that the dome shape and tapered shape have a more uniform surface. As a result, the implant 10 is able to conform more closely to the morphology of the vertebral end plates it comes into contact with. In addition, the zigzag pattern 380 on the outer caps 376 facilitates potential stretch. More specifically, stitches may be placed at every direction-change according to the zigzag pattern 380, thus providing the desired flexibility/elasticity in the outer caps 376 that is needed when used to cover the top and bottom contours of the spacer 12.

Although the outer caps 376 of the encapsulating jacket 14 are described herein as having a zigzag pattern or flexible fabric, it will be appreciated that any type of material that imparts potential stretch or expansion may be used without departing from the scope of the present invention. It will also be appreciated that the outer caps 376 may be rigid and may not be designed to allow for expansion without departing from the scope of the present invention. In all instances, the outer caps 376 of the encapsulating jacket 14 cover the top and bottom of the spacer 12 and contribute to the encapsulation/containment of the implant 10 within the intervertebral disc space.

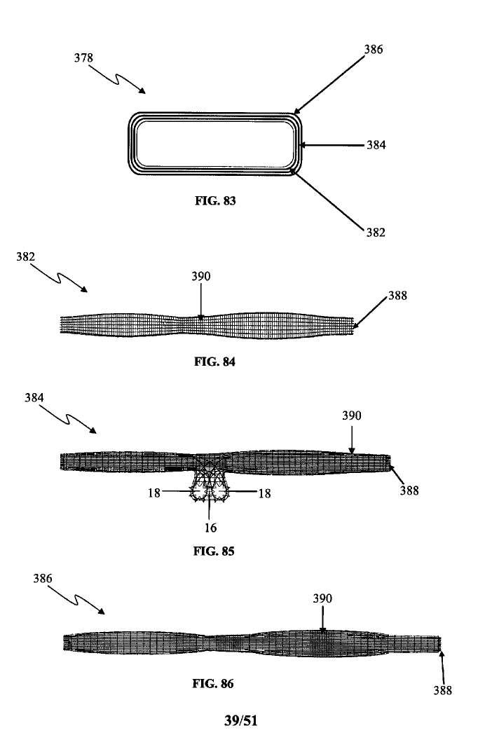

FIGS. 83-86 illustrate an example of a circumferential barrier 378 of the encapsulating jacket 14 according to one embodiment of the present invention. As shown, the circumferential barrier 378 may be comprised of three circumferential layers: an inner layer 382, a middle layer 384 (with attachment flange 16), and an outer layer 386. The three circumferential layers 382-386 may have longitudinal load-bearing threads 388 and vertical load-bearing threads 390 as shown in FIGS. 84-86. By consisting of longitudinal and vertical load-bearing threads 388, the three circumferential layers 382-386 are designed to radially contain the spacer 12, while sustaining the axial pressure from the adjacent vertebral bodies. By consisting of vertical load-bearing threads 390, the circumferential barrier holds the layer regions of the spacer 12 together. In this way, the circumferential barrier 378 retains the core shape of the spacer 12 and reinforces the structure of the implant 10, thereby restoring the height of the intervertebral disc space.

According to the example of the circumferential barrier 378 described herein, the inner circumferential layer 382 primarily contains vertical load-bearing threads in order to hold the layer regions of the spacer 12 tightly together, as shown in FIG. 84. To hold the spacer 12 together, the top layer region of the spacer is stitched to the top edge of the inner circumferential layer 382 and the bottom layer region of the spacer is stitched to the bottom edge of the inner circumferential layer 382. As shown in FIG. 85, the middle circumferential layer 384 primarily contains longitudinal load bearing threads 390 to radially contain the spacer. As shown in FIG. 86, the outer circumferential layer 386 primarily contains both longitudinal and vertical load bearing threads 388, 390 to provide both axial support and to secure attachment for the top and bottom layers of the spacer 12, respectively. Although shown and described as having three circumferential layers 382-386, it will be appreciated that the circumferential barrier 378 of the jacket 14 is not limited to this number of layers and may contain any number of layers without departing from the scope of the present invention. In all instances, the circumferential barrier 378 will retain the height, shape and structure of the implant 10.

By way of example only, the middle circumferential layer 384 may include an attachment flange 16 having apertures 18 formed therein, as shown in FIG. 85. The flange 16 extends from the middle layer 384 such that it can be used for affixing the encapsulating jacket 14 to an adjacent anatomic structure (e.g. an adjacent vertebral body) to maintain the implant 10 in position before the embroidery becomes encapsulated with scar tissue. Apertures 18 may be optionally provided in the flange 16 to accommodate anchors, such as screws or any other suitable affixation elements (e.g. nails, staples, bone anchors, etc.), which secure the implant 10 to the adjacent spinal vertebrae. Although the encapsulating jacket 14 is shown as having only one attachment flange 16, it will be appreciated that this number is set forth by way of example only and that the number of flanges may be increased or decreased without departing from the scope of the present invention. Furthermore, it will be appreciated that the attachment flange 16 is not limited to the middle circumferential layer 384 and may be attached to any component of the circumferential barrier 378 and/or encapsulating jacket 14 without departing from the scope of the present invention.

Although shown as fully encapsulating the spacer 12, it will be appreciated that the jacket 14 may partially encapsulate the spacer 12 (i.e. with one or more apertures formed in the jacket 14 allowing direct access to the core 12) without departing from the scope of the present invention. Moreover, the various components of the encapsulating jacket 14 may be attached together via an embroidery process, or any other suitable technique. In addition, the various layers and/or components of the spacer 12 may be attached or unattached to the encapsulating jacket 14 without departing from the scope of the present invention.

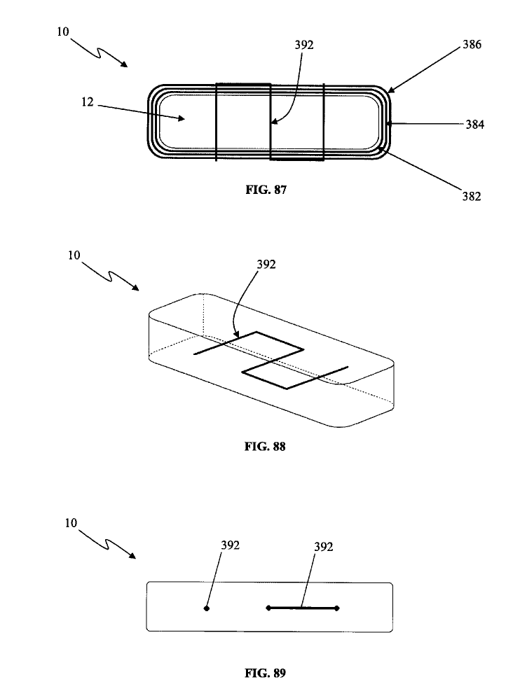

In addition, reinforced stitching 392 may be added throughout the implant 10, as shown in FIGS. 87-89, in order to prevent anterior-posterior and/or medial-lateral bulging under pressure. The reinforced stitching 392 may be constructed from the same material as the implant 10, or may be made from different materials, including but not limited to polyester, metal wire, tantalum, platinum/iridium, barium loaded polymer, and/or fiber wire. Using a separate material (e.g. metal fibers) to construct the reinforced stitching 392 may serve a dual purpose: to prevent midline bulging and to act as a radio-opaque marker. It will be appreciated that the placement of the reinforced stitching 392 within the implant 10 in FIGS. 87-89 is shown by way of example only and may be changed without departing from the scope of the present invention.

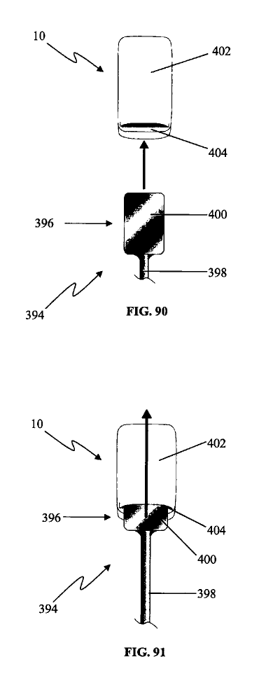

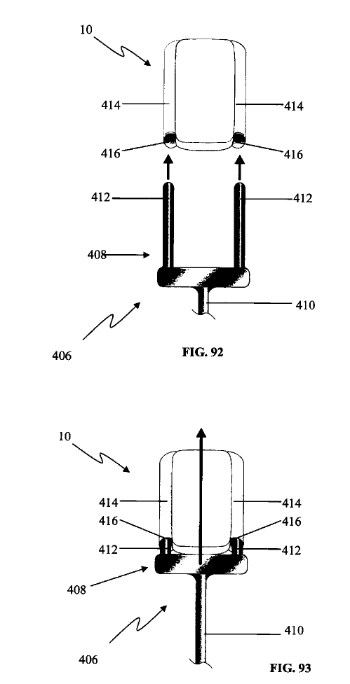

FIGS. 90 & 91 illustrate a first example of an inserter 394, used for inserting an implant 10 into an intervertebral disc space according to the present invention. The inserter 394 is designed to releasably maintain the implant 10 in the proper orientation for insertion. The implant 10 may be introduced into an intervertebral disc space while engaged with the inserter 394 and thereafter released from the inserter 394. Preferably, the inserter 394 may include a distal engagement region 396 and an elongated handling member 398. The inserter 394 may be composed of any material suitable for inserting an implant 10 into an intervertebral disc space, including but not limited to metal (e.g. titanium, stainless steel, etc.), ceramic, and/or polymer compositions. According to this particular embodiment, the distal engagement region 396 is comprised of an insertion plate 400. The insertion plate 400 is generally planar rectangular in shape, but may take the form of any geometric shape necessary to interact with the implant 10, including but not limited to generally oval, square, and triangular. The handling member 398 is generally cylindrical in shape. The handling member 398 allows a clinician to manipulate the tool during an implant insertion procedure.

In order to facilitate engagement with the inserter 394, the implant 10 may include a pocket 402. By way of example only, the pocket 402 may be an extra layer of embroidered fabric attached to three of the four sides of the implant 10, leaving an opening 404 for insertion of the insertion plate 400. The insertion plate 400 engages with the implant 10 by sliding into the pocket 402. Although slideable engagement is described herein, any suitable means of engagement may be used to engage the insertion plate 400 with the implant 10, including but not limited to a threaded engagement, snapped engagement, hooks, and/or compressive force. Once the insertion plate 400 is fit into place within the pocket 402 of the implant 10, the inserter 394 releasably maintains the implant 10 in the proper orientation for insertion. The implant 10 may then be introduced into an intervertebral disc space while engaged with the inserter 394 and thereafter released so that the inserter 394 may be removed from the disc space and operative corridor. The implant 10, having been deposited in the intervertebral disc space, facilitates normal spinal functionality over time by maintaining a restored intervertebral disc height (due to the structural and load-bearing capabilities of the implant 10) as well as retaining a normal range of motion.

FIGS. 92 & 93 illustrate second example of an inserter 406, used for inserting an implant 10 into an intervertebral disc space according to the present invention. The inserter 406 may include a distal engagement region 408 and an elongated handling member 410. In this example, the distal engagement region 408 is comprised of two insertion prongs 412. Preferably, the insertion prongs 412 are generally cylindrical in shape, but may take the form of any geometric shape necessary to interact with the implant 10. In order to facilitate engagement with the insertion prongs 412, the implant 10 may have attached side pockets 414. By way of example only, the side pockets 414 may be made of embroidered fabric attached to each side of the spacer 10 with openings 416 for insertion of the insertion prongs 412.

The insertion prongs 412 engage with the implant 10 by sliding into the side pockets 414. Although slideable engagement is described herein, any suitable means of engagement may be used to engage the insertion prongs 412 with the implant 10, including but not limited to a threaded engagement, snapped engagement, hooks, and/or compressive force. Once the insertion prongs 412 are inside the side pockets 414 of the implant 10, the inserter 406 releasably maintains the implant 10 in the proper orientation for insertion. The implant 10 may then be introduced into an intervertebral disc space while engaged with the inserter 406 and thereafter released. It will be appreciated that the number of insertion prongs 412 on the inserter 406 (and corresponding pockets 414 on the implant 10) is set forth by way of example only and may be increased or decreased without departing from the scope of the present invention. In all instances, the implant 10, having been deposited in the intervertebral disc space, facilitates normal spinal functionality over time by maintaining a restored intervertebral disc height (due to the structural and load-bearing capabilities of the implant 10) as well as retaining motion. It will be appreciated that the inserter 406 of the present invention is not limited to interaction with the implant 10 disclosed herein, but rather may be dimensioned to engage any surgical implant.

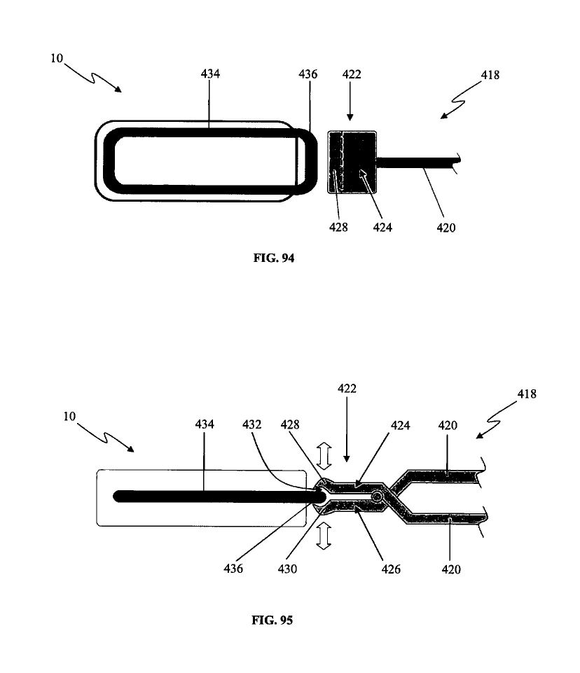

FIGS. 94 & 95 illustrate a third example of an inserter assembly 418, used for inserting an implant 10 into an intervertebral disc space according to the present invention. According to this example, the inserter 418 may include a pair of elongated handling members 420 and a distal engagement region consisting of a clamping mechanism 422. The handling members 420 are generally cylindrical in shape and allow a clinician to manipulate the tool during an implant insertion procedure. The inserter 418 may be composed of any material suitable for inserting an implant 10 into an intervertebral disc space, including but not limited to metal (e.g. titanium, stainless steel, etc.), ceramic, and/or polymer compositions.

The clamping mechanism 422 is comprised of a pair of clamping plates, an upper clamping plate 424 and a lower clamping plate 426, each having a curved “C”-shaped groove at its distal end 428, 430, respectively. The clamping plates 424, 426 are generally planar rectangular in shape and parallel to one another. The grooved ends 428, 430 of the clamping plates 424, 426 are oriented such that each respective “C” shape faces one another, thereby forming a cradle 432 for engagement with the implant 10. Preferably, the grooved ends 428, 430 grasp a wireframe 434 contained in the implant 10.

In order to provide an attachment point for grasping by an inserter 418, the implant 10 of this example is provided with a wireframe 434 that is sufficiently large and rigid. The wireframe 434 of the implant 10 may be composed of any material suitable for engagement with an inserter 418, including but not limited to metal (e.g. titanium, stainless steel, etc.), ceramic, and/or polymer compositions. A wireframe 434 that is composed of metal fibers or any other type of radio-opaque material may also serve as a radio-opaque marker. As shown in FIG. 94, the wireframe 434 is a cylindrical frame that runs along the inside perimeter of the rectangular implant 10 and extends past one side of the implant 10 to provide a rail 436 for the inserter 418 to grab onto. Furthermore, the wireframe 434 adds stiffness to the implant 10 to help facilitate insertion.

As shown in FIG. 95, the grooved ends 428, 430 of the clamping plates 424, 426 of the inserter 418 engage with the implant 10 by clamping onto the rail 436 of the wireframe 434. More specifically, the grooved end 428 of the upper clamping plate 424 encloses on the top part of the rail 436 and the grooved end 430 of the lower clamping plate 426 encloses on the bottom part of the rail 436. Once the rail 436 is clamped within the cradle 432 of the inserter 418, the inserter releasably maintains the implant 10 in proper orientation for insertion. The implant 10 may then be introduced into an intervertebral disc space while engaged with the inserter 418 and thereafter released. The implant 10, having been deposited in the intervertebral disc space, facilitates normal spinal functionality over time by maintaining a restored intervertebral disc height (due to the structural and load-bearing capabilities of the implant 10) as well as retaining a normal range of motion.

Although a clamping engagement is described herein, any suitable means of engagement may be used to engage the clamping plates 424, 426 of the inserter 418 with the wireframe 434 of the implant 10, including but not limited to a threaded engagement, slideable engagement, snap engagement, hooks, and/or compressive force. It will be appreciated that the generally rectangular shape of the wireframe 434 is set forth by way of example only and that the shape may be changed without departing from the scope of the present invention. For instance, the wireframe 434 may be any of generally square, oval, elliptical, trapezoidal, and polygonal shape to better conform to the shape of the implant 10. In addition, the shape of the inserter 418 may be changed to accommodate a different shape of the wireframe 434 in order to facilitate engagement.

Although shown and described in this third example as having a wireframe 434, the implant 10 may not include a wireframe 434 and the inserter 418 may grasp the entire implant 10 during insertion without departing from the scope of the present invention. In all instances, the implant 10, having been deposited in the intervertebral disc space, facilitates normal spinal functionality over time by maintaining a restored intervertebral disc height (due to the structural and load-bearing capabilities of the implant 10) as well as retaining motion. It will be appreciated that the inserter 418 of the present invention is not limited to interaction with the implant 10 disclosed herein, but rather may be dimensioned to engage any surgical implant.

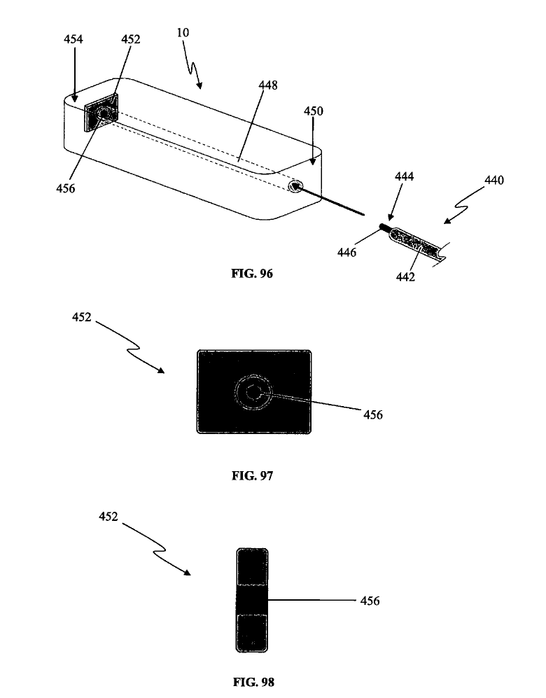

FIGS. 96-98 illustrate a fourth example of an inserter assembly 440, used for inserting an implant 10 into an intervertebral disc space according to the present invention. The inserter 440 may be composed of any material suitable for inserting an implant 10 into an intervertebral disc space, including but not limited to metal (e.g. titanium, stainless steel, etc.), ceramic, and/or polymer compositions. The inserter 440 includes an elongated handling member 442 and a distal engagement region. The distal engagement region 444 is comprised of a generally cylindrical-shaped threaded engagement feature 446. The handling member 442 is generally cylindrical in shape and allows a clinician to manipulate the tool during an implant insertion procedure. Although shown and described as having a cylindrical shape, the inserter 440 may have a handling member 442 and an engagement feature 446 that is any number of suitable shapes, including but not limited to rectangular or triangular.

In order to facilitate engagement with the inserter 440, the implant 10 includes an aperture 448 at the proximal side 450 of the implant 10. The implant 10 also includes an engagement plate 452 at the distal side 454 of the implant 10. The aperture 448 extends inwardly from the proximal side 450 in a generally perpendicular fashion relative to the proximal side 450. The aperture 448 may extend through the implant 10 until it reaches the engagement plate 452 at the distal side 454 of the implant 10. Preferably, the aperture 448 has a diameter larger than the inserter 440 such that the inserter 440 may slidably engage with the implant 10 and fit within the aperture 448. Although shown as having a generally circular cross-section, it will be appreciated that the aperture 448 may be provided having any number of suitable shapes or cross-sections to facilitate the inserter 440, including but not limited to rectangular or triangular.

The engagement plate 452 is generally rectangular in shape and may be composed of metal (e.g. titanium, stainless steel, etc.), ceramic, and/or polymer compositions. If composed of metal fibers or any other type of radio-opaque material, the engagement plate 452 may also serve as a radio-opaque marker. To contain the engagement plate 452, the implant 10 may include a pocket at the distal side 454. Alternatively, the engagement plate 452 may be stitched under the outer layer or encapsulating jacket of the implant 10 at its distal side 454. The engagement plate 452 includes at its center a threaded hole 456 to provide a point of attachment for the engagement feature 446 of the inserter 440. The threaded hole 456 on the engagement plate 452 matches the threaded engagement feature 446 on the inserter 440 so that they can be threadably attached to one another. Other methods of creating a gripping surface are contemplated including but not limited to knurling or facets.

The inserter 440 engages with the implant 10 by entering the aperture 448 on the proximal side 450 of the implant 10. The inserter 440 slides through the aperture 448 until it reaches the distal side 454 of the implant 10 and comes in contact with the engagement plate 456. At that time the implant 10 and inserter 440 are slidably engaged with one another. Although slideable engagement is described herein, any suitable means of engagement may be used, including but not limited to a threaded engagement, snapped engagement, hooks, and/or compressive force. Before the clinician can manipulate the combined implant 10 and inserter 440, they must be releasably secured together. In order to secure the implant 10 onto the inserter 440, the engagement feature 446 of the inserter 440 is threaded through the hole 456 of the engagement plate 452 and securely fastened.

Once securely engaged, the inserter 440 releasably maintains the implant 10 in proper orientation for insertion. The implant 10 may then be introduced into an intervertebral disc space while engaged with the inserter 440 and thereafter released from the inserter. More specifically, the inserter 440, while threaded to the implant 10, pushes against the engagement plate 452 at the distal end 454 of the implant 10. The engagement plate 452 adds stiffness to the implant 10 at its distal end 454, thereby providing a rigid edge for leading the implant 10 through the insertion corridor and into the intervertebral space. In this way, the engagement plate 452 helps maintain the shape of the implant 10 during the process of insertion. After inserting the implant 10, the inserter 440 is unfastened from the engagement plate 452 and removed. The implant 10, having been deposited in the intervertebral disc space, facilitates normal spinal functionality over time by maintaining a restored intervertebral disc height (due to the structural and load-bearing capabilities of the implant 10) as well as retaining a normal range of motion.

Although shown and described as having a generally rectangular shape, it will be appreciated that the engagement plate 452 may be provided in any suitable shape to conform with the implant 10, including but not limited to, square, oval, elliptical, trapezoidal, and polygonal shape. It will be understood that the inserter assembly 440 and engagement plate 452 of the present invention is not limited to interaction with the implant 10 disclosed herein, but rather may be dimensioned to engage any surgical implant. It will also be appreciated that the implant 10 may not include an engagement plate 452. By way of example only, the inserter 440 may slidably engage with the implant 10 through the aperture 448 without threadably engaging with an engagement plate 452 at the distal side. In all instances, the implant 10, having been deposited in the intervertebral disc space, facilitates normal spinal functionality over time by maintaining a restored intervertebral disc height as well as retaining motion.

While lateral insertion is described herein, it will be appreciated that the textile-based spinal implant of the present invention may be inserted through any suitable surgical technique, including but not limited to anterior insertion, antero-lateral insertion, or posterior insertion. It will be understood that the implant may be used in a variety of surgical applications and is not limited to spine surgery. When used in spinal surgery, the implant may be applied to any level of the spine, including but not limited to the lumbar, thoracic, cervical, partially fused sacral, and iliosacral joints.

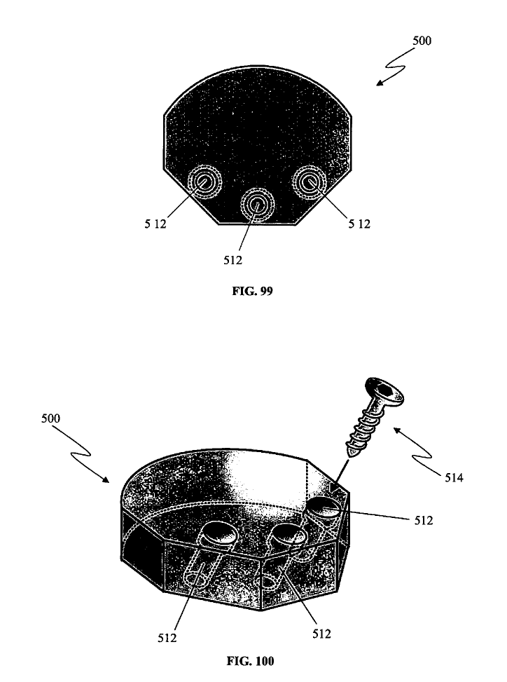

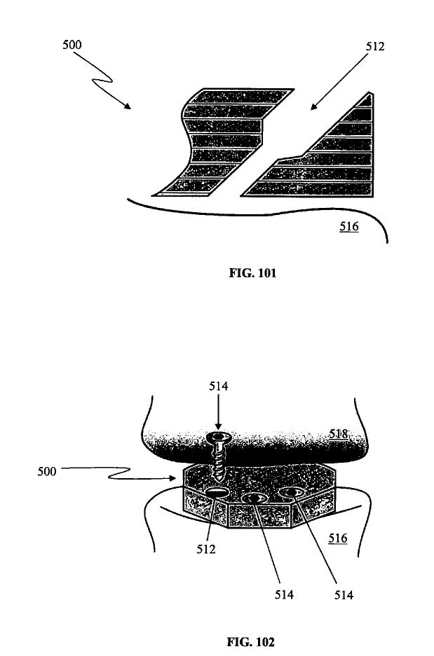

By way of example only, the following embodiments described below provide a textile-based spinal implant 500 that may be inserted through anterior insertion. FIGS. 99-102 collectively illustrate an example of an implant 500 that may be inserted through anterior insertion, according to one embodiment of the present invention. Although not shown, the implant 500 may comprise a textile-based spacer disposed within an encapsulating jacket. The implant 500 may incorporate one or more or all of the features described above, including but not limited to, the anatomical dome and/or tapered shapes described above. According to this example, the implant 500 includes screw holes 512 or apertures dimensioned to receive screws 514 or other affixation elements (e.g. nails, staples, etc.). Although shown and described herein as having angled screw holes 512 each with an entry point extending from the top surface of the implant 500, it will be appreciated that the angled screw holes 512 may be provided each having an entry point on any portion of the outwardly facing surface(s) of the implant 500 (e.g. the anterior face as shown in FIG. 102. This would require less distraction than having the screw holes 512 with entry points on top of the implant as shown.

The implant 500 is inserted in the intervertebral disc space between two adjacent vertebrae 516, 518 of the spine. Once inserted, the implant 500 is screwed directly into position in the intervertebral disc space. The screws 514 pass through the screw holes 512 of the implant 500. The screws 514 are then drilled into the inferior vertebra 516 to secure the implant 500 in position. Although the implant 500 is shown as having three screw holes 512 to accommodate three screws 514, it will be appreciated that any number of screw holes 512 in the implant 500 and screws 514 may be used to affix the implant 500 in situ. Furthermore, the implant 500 may be provided in any number of suitable dimensions depending upon the surgical application and patient pathology.

As shown in FIGS. 100 & 101, the screw holes 512 within the implant 500 are angled relative to the bone surface 516 (i.e. not perpendicular) such that there is an angle of fixation when the screw 514 is drilled into the inferior vertebra 516 to secure the implant 500 in situ. Angled screw holes 512 within the implant 500 are desirable and advantageous when securing the implant 500 inside an intervertebral disc space because they allow for proper affixation of the screws 514 when there is little access to the inferior vertebra 516 due to the vertical alignment of the adjacent vertebrae. Accordingly, if there is no space for head-on access (or a direct perpendicular path) for the screws 514 to affix the implant 500 to the inferior vertebra 516, the angled screw holes 512 allow the implant 500 to be securely affixed to the inferior vertebra 516 at an angle. It will be appreciated that the screw holes 512 may be angled to any degree in order to facilitate any type of affixation without departing from the scope of the present invention. Although described and shown as having angled screw holes 512, it will be appreciated that the screw holes 512 of the implant 500 may not be angled depending upon the surgical application and patient pathology.

Furthermore, although described herein largely in terms of affixing the implant 500 to the inferior vertebra 516, it will be understood that the implant 500 may be attached to the superior vertebra 518 without departing from the scope of the present invention. This may apply to any embodiment of the implant 500 described herein. In all instances, it is understood that whether the implant 500 is affixed to the inferior vertebra 516 or if the implant 500 is affixed to the superior vertebra 518, the implant 500 will be situated in the intervertebral disc space either way and will result in the repair/reconstruction of the degenerative joint.

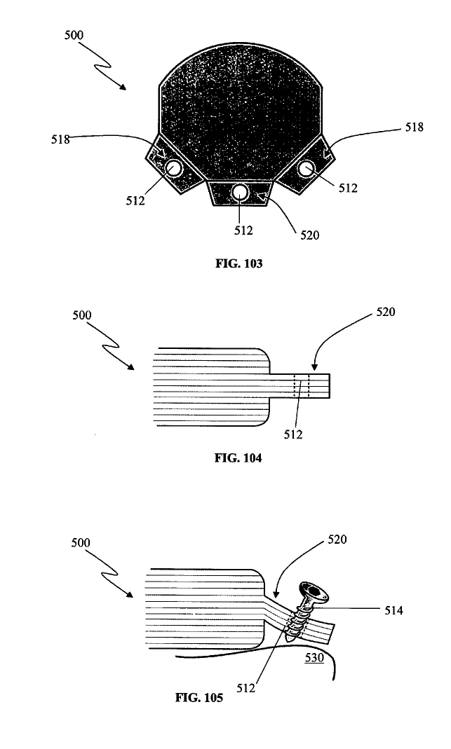

FIGS. 103-105 collectively illustrate an example of an implant 500 according to a another embodiment of the present invention. According to this example, the implant 500 includes attachment flanges 520 positioned on the side of the implant 500, as shown in the side cross-sectional view of FIGS. 104 & 105. Each attachment flange 520 may have a screw hole 512 dimensioned to receive screws 514 or other affixation elements (e.g. nails, staples, etc.). The implant 500 is inserted between two bone surfaces of a joint (e.g. an intervertebral disc space between two adjacent vertebrae) to prevent bone-on-bone contact. Once the implant 500 is inserted, screws 514 pass through the screw holes 512 in the attachment flanges 520 of the implant 500. The screws 514 are then drilled into the bone 530 to secure the implant 500 in position. As described above in detail, the screw holes 512 may or may not be angled in order to facilitate affixation of the implant 500 to the adjacent bone surface 530.

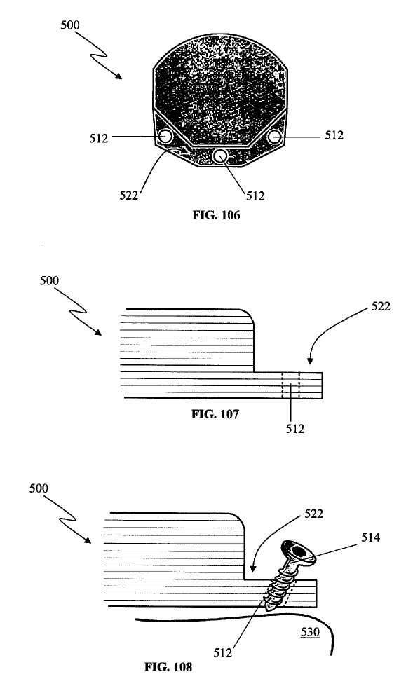

Although the implant 500 is shown as having three attachment flanges 520 each having a screw hole 512 to accommodate three screws 514, it will be appreciated that any number of attachment flanges 520, screw holes 512, and screws 514 may be used to affix the implant 500 in situ. It will also be appreciated that the location/placement of the attachment flange or flanges 520 on the implant 500 may vary without departing from the scope of the present invention. By way of example only, FIGS. 106-108 illustrate an implant 500 including one attachment flange 522 that has three screw holes 512 dimensioned to receive screws 514 or other affixation elements (e.g. nails, staples, etc.). In addition, the attachment flange 522 is located at the base of the implant 510 as shown in the side-cross sectional view of FIGS. 107 & 108.

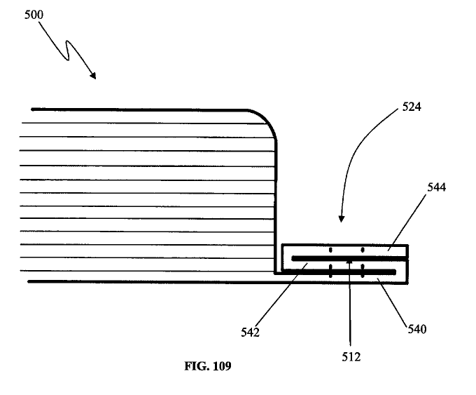

In a further embodiment of the present invention, the attachment flange 524 may be comprised of multiple layers 540-544 folded on top of one another, as shown in FIG. 109 (instead of a single layer extending from the implant 500 as shown in FIGS. 103-108). In all cases, it will be understood that the attachment flange 520, 522, 524 of the implant 500 results in the implant 500 being secured within the joint, thereby repairing/reconstructing the degenerative joint by preventing bone-on-bone contact and preserving the natural motion of the joint.

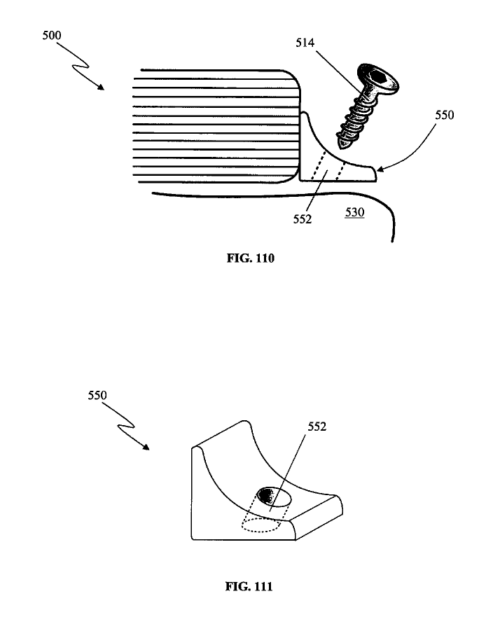

FIGS. 110 & 111 collectively illustrate an example of an implant 500 according to another embodiment of the present invention. According to this example, the implant 500 is attached to a fixation buttress 550 made out of metal (e.g. titanium), ceramic, plastic, and/or polymer compositions. The fixation buttress 550 includes a screw hole 552 dimensioned to receive screws 514 or other affixation elements (e.g. nails, staples, etc.). Similar to the second example described above, the implant 500 is inserted between two bone surfaces of a joint (e.g. an intervertebral disc space between two adjacent vertebrae) to prevent bone-on-bone contact. Once the implant 500 with fixation buttresses 550 is inserted, screws 514 pass through the screw holes 552 of the fixation buttresses 550. The screws 514 are then drilled into the bone 530 to secure the implant 500 in position. As described above in detail, the screw holes 552 may or may not be angled in order to facilitate affixation of the implant 500 to the adjacent bone surface 530. It will be appreciated that any number fixation buttresses 550, screw holes 552, and screws 514 may be used to affix the implant 500 in situ. In all cases, it will be understood that the implant 500 prevents bone-on-bone contact while advantageously preserving the natural motion of the joint.

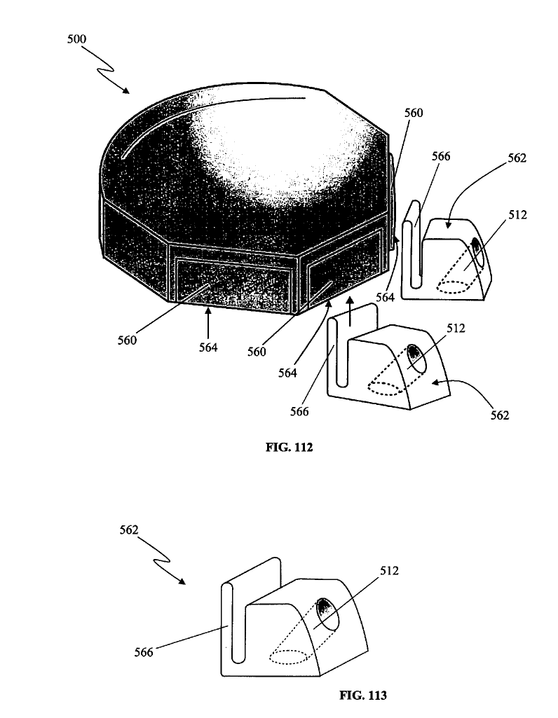

FIGS. 112 & 113 collectively illustrate an example of an implant 500 according to another embodiment of the present invention. According to this example, the implant 500 has pockets 560 in order to facilitate clip-on fixation buttresses 562. By way of example only, the pockets 560 on the implant 500 are extra layers of embroidered fabric attached to the side of the implant 500 with openings 564 for engagement with the clips 566 of the clip-on fixation buttresses 562. The clip-on fixation buttresses 562 may be composed of any suitable material, including but not limited to metal (e.g. titanium), ceramic, plastic, or polymer compositions.

The implant 500 is inserted between two bone surfaces of a joint (e.g. an intervertebral disc space between two adjacent vertebrae) to prevent bone-on-bone contact. Once the implant 500 is inserted, the clips 566 of the clip-on fixation buttresses 562 engage with the implant 500 by sliding into the openings 564 of the pockets 560 on the implant 500. The clips 566 secure the attachment of the fixation buttresses 562 to the implant 500. Next, screws 514 pass through the screw holes 512 of the fixation buttresses 562. The screws 514 are then drilled into the bone to secure the implant 500 in position. As described above in detail, the screw holes 512 may or may not be angled in order to facilitate affixation of the implant 500 to the adjacent bone surface. It will be appreciated that any number of fixation buttresses 562, screw holes 512, and screws 514 may be used to affix the implant 500 in situ. In all cases, it will be understood that the implant 500 prevents bone-on-bone contact while advantageously preserving the natural motion of the joint.

Furthermore, although described as inserting the implant 500 first and then attaching the clip-on fixation buttresses 562 second, it will be appreciated that the implant 500 and clip-on fixation buttresses 562 may be inserted in any order without departing from the scope of the present invention. For example, the clip-on fixation buttresses 562 may be inserted first and the implant 500 attached second, the clip-on fixation buttresses 562 may be attached to the implant 500 before insertion and both the clip-on fixation buttresses 562 and implant 500 be inserted simultaneously, or any other suitable method of inserting the implant 500 and attachable clip-on fixation buttresses 562 into the target space may be used. In all cases, it will be understood that the clip-on fixation buttresses 562 attached to the implant 500 and inserted between two bone surfaces result in the implant 500 being secured within the target space and the degenerative joint being repaired/reconstructed due to the implant 500 preventing bone-on-bone contact and preserving the natural motion of the joint.

As evidenced by the foregoing, the textile-based implant 10, 500 of the present invention is suitable for use in a variety of surgical applications, including but not limited to spine surgery. When applied to spinal surgery and implanted into an intervertebral disc space, the tapered and/or contoured shape of the textile-based implant 10, 500 of the present invention advantageously forces the adjacent vertebral bodies into an angled relationship, thereby restoring (partially or fully) the natural curvature of the spine at that vertebral level. More specifically, the tapered cross-sectional shape of the implant 10, 500 is designed to match the natural lordotic and/or kyphotic angles in any given region of the spine (i.e. lordosis in the cervical and lumbar regions of the spine and kyphosis in the thoracic region of the spine). The textile construction of the tapered textile-based implant 10, 500 of the present invention is generally compliant and thereby advantageously restores and/or improves spinal motion in that vertebral level relative to traditional fusion surgery. In other words, the compliant nature of the tapered textile-based implant 10, 500 provides the required flexibility and elasticity to support the full range of physiologic moments, as opposed to fusion surgery which forms a boney bridge between adjacent vertebral bodies. The porosity and biocompatibility of the textile-based implant 10, 500 facilitates tissue ingrowth throughout part or all of the implant (as desired), which helps to secure and encapsulate the tapered textile-based implant 10, 500 in the intervertebral space.

In use, the textile-based surgical implant 10, 500 is dimensioned for insertion into an intervertebral space. To accomplish this, an operative corridor is established between the outside of the skin to the surgical target site (e.g. intervertebral disc space). Various steps and methods of establishing an operative corridor are well known in the art, including wound incision, tissue distraction, tissue retraction, etc. The next step is to at least partially clean out the disc space, for example by total or partial discectomy, to create a space for insertion of the implant 10, 500. The implant is then implanted using one of the various examples of insertion devices and techniques described herein and then optionally secured to at least one of the adjacent vertebral bodies using a fixation element. After the implant is secure, the operative corridor and wound incision are closed, completing the procedure.

It will be appreciated that the implant 10, 500 may incorporate one or more or all of the features described herein and any combination thereof without departing from the scope of the invention. It will also be appreciated that the features described above can be applied to any of the embodiments disclosed herein. While this invention has been described in terms of a best mode for achieving this invention's objectives, it will be understood by those skilled in the art that variations may be accomplished in view of these teachings without deviating from the spirit or scope of the invention.

1. A textile-based spinal implant dimensioned to be implanted within an intervertebral space, comprising:
a core comprising a plurality of textile layer regions stacked on top of one another to form a spacer element; and
a textile jacket encapsulating the core, the jacket having a pair of opposing vertebral contact surfaces, each vertebral contact surface configured to contact a surface of a vertebral body.

2. The implant of claim 1, wherein the plurality of textile layer regions are formed from at least one of a plurality of individual textile layer elements consecutively stacked on top of one another, a plurality of hingedly connected individual textile layer regions folded on top of one another, and a single continuous textile sheet folded upon itself to form a plurality of stacked textile layer regions.

3. The implant of claim 1, wherein the plurality of stacked textile layer regions are coupled together by at least one of supplemental stitching and adhesives.

4. The implant of claim 1, wherein one or more of the plurality of textile layer regions is contoured such that the spacer element has a cross-section having a pair of opposing non-parallel sides, the non-parallel sides corresponding to the vertebral contact surfaces of the textile jacket.

5. The implant of claim 4, wherein the opposing non-parallel sides are at least one of partially angled relative to one another, generally tapered relative to one another, arranged in a stair step fashion, and curved relative to one another.

6. The implant of claim 4, wherein the one or more textile layer regions is contoured by repeatedly overstitching different sections of the textile layer region using thread of the same thickness to form sections varying in at least one of height and stitch density.

7. The implant of claim 4, wherein the one or more textile layer regions is contoured by stitching different sections of the textile layer region using threads of varying thickness to form sections having varying height.

8. The implant of claim 4, wherein the one or more textile layer regions is contoured by stacking fabric layers having at least one of different sizes and shapes consecutively on top of one another to form sections having different heights.

9. The implant of claim 4, wherein the one or more textile layer regions is contoured by stitching different sections of the textile layer region using sections of different yarn spacing to form sections having different heights.

10. The implant of claim 1, wherein the core is provided with at least one internal radio-opaque marker to enable visualization of the implant after implantation using fluoroscopy.

11. The implant of claim 10, wherein the radio-opaque marker is at least one of an elongated cylinder, a short cylinder, a bead, and an annular ring.

12. The implant of claim 1, wherein the encapsulating jacket has at least one expandable outer cap and a circumferential barrier effective to reinforce the shape of the spacer element.

13. The implant of claim 1, further comprising reinforced stitching throughout the implant, the reinforced stitching effective to prevent bulging of the implant after insertion into an intervertebral space.

14. The implant of claim 1, wherein the implant is dimensioned for insertion into the intervertebral space from at least one of a lateral aspect of a spine and an anterior aspect of a spine.

15. A system for performing spinal surgery, comprising:
a textile-based spinal implant, the implant including a core comprising a plurality of textile layer regions stacked on top of one another to form a spacer element, an encapsulating jacket, and an engagement element dimensioned to facilitate engagement with an insertion device;
an insertion device including a proximal handle portion to allow for user manipulation and a distal engagement region dimensioned to engage with the engagement element of the implant; and
a fixation element configured to securely affix the implant to at least one vertebral body.

16-19. (canceled)

20. The system of claim 15, wherein the engagement element comprises at least one pocket provided on the encapsulating jacket.

21. The system of claim 20, wherein the distal engagement region of the insertion device comprises an insertion plate dimensioned to be received within the at least one pocket.

22. The system of claim 15, wherein the engagement element comprises a wireframe provided within the implant, at least a portion of the wireframe at least partially protruding from the implant.

23. The system of claim 22, wherein, wherein the distal engagement region of the insertion device comprises a clamping mechanism configured to engage the portion of the wireframe at least partially protruding from the implant.

24. The system of claim 15, wherein the engagement element comprises an engagement plate positioned at a distal end of the implant and including a threaded aperture, said implant further comprising an aperture extending from a proximal end of the implant to the engagement plate.

25. The system of claim 24, wherein the distal engagement region of the insertion device comprises a threaded engagement feature configured to threadedly engage the threaded aperture of the engagement plate.

26. The system of claim 15, wherein the fixation element comprises at least one of a bone screw, nail, tack, suture, and adhesive.

27. The system of claim 15, wherein the fixation element comprises a fixation buttress and a screw.

28. The system of claim 27, wherein the fixation buttress is at least one of attached and clip-on.

29. A method for performing spinal surgery, comprising:
providing a textile-based spinal implant the implant including a core comprising a plurality of textile layer regions stacked on top of one another to form a spacer element, an encapsulating jacket, and an engagement element dimensioned to facilitate engagement with an insertion device;
engaging said engagement element to an insertion device; and
inserting said spinal implant into a surgical target site, and thereafter releasing said implant from said insertion device.

30-33. (canceled)
